(19) United States
(12) Patent Application Publication
(10) Pub. No.: US 2023/0305020 A1
(43) Pub. Date: Sep. 28, 2023

(54) Assays for Assessment of Immune Function and Antibody Survey

(71) Applicant: Acari Bio, Inc., Walnut, CA (US)

(72) Inventors: Jeroen Bekaert, Walnut, CA (US); Michal Tal, Cupertino, CA (US); Rachel E. Salomon-Shulman, Redwood City, CA (US); Hanna Ollila, East Palo Alto, CA (US)

(21) Appl. No.: 18/007,042

(22) PCT Filed: Jul. 29, 2021
(86) PCT No.: PCT/US2021/043798

§ 371 Date: Jan. 26, 2023

Related U.S. Application Data
(60) Provisional application No. 63/058,482, filed on Jul. 29, 2020.

(51) Int. Cl. G01N 33/68; G01N 33/569

(52) U.S. Cl. CPC G01N 33/6854; G01N 33/56983; G01N 2469/20

(57) ABSTRACT

Provided are assays for functional immune assessment, immune element survey, and diagnostic methods using cultured cells. The immune functional assays enable assessment of subject immune function against a pathogen, utilizing antigen baits and target constructs. Bead based antigen baits provide a robust and facile method of assessing protective immune responses for pathogens, including systems for assessing immune function against SARS-CoV-2. Also provided are immune element surveys that enable profiling of immune elements deployed against a selected pathogen or antigen. Cell culture methods provide a novel and facile tool for assessment of immune function and diagnostic tools for detecting prior or current infection by pathogens. The assays may advantageously be combined for full spectrum analysis of a subjects immune response.

### CROSS-REFERENCE TO RELATED APPLICATIONS

This application claims the benefit of priority to U.S. Provisional Patent Application Ser. No. 63/058,482 entitled “Functional Multiplexed Immune Profiling,” filed Jul. 29, 2020, the contents of which are hereby incorporated by reference.

### BACKGROUND OF THE INVENTION

Immune system function is a key component of human health. Immune functions defend the body against pathogens, while immune system dysfunction underlies conditions such as autoimmune disease and allergies. These functions and pathologies are mediated by a complex suite of immune elements, including antibodies, T-cells, innate immune effectors, and others. Given the importance of the immune system to human health, there is a need in the biomedical arts for tools to assess immune function and to survey the immune elements that are active in an immune response of interest.

In a first aspect, there is a need in the art for assessing the effectiveness of a subject's immune response to a particular pathogen. Specifically, it is of critical importance to determine whether a subject has an effective immune response against a harmful virus or other pathogen. A stark example of this need is presented by the ongoing COVID-19 pandemic caused by the SARS-CoV-2 virus. The worldwide battle against this virus has required enormous expenditures on vaccine design, clinical trials, and deployment. At the individual level, it is important to determine if a subject has immunity or is susceptible to the virus and its evolving variants. At a systemic level, it is of critical importance to determine if a particular vaccine will protect against the virus and its evolving variants. Currently available tools for assessing immunity to the virus are expensive or inadequate. For example, various diagnostic kits are available which can detect the presence of serum antibodies against SARS-CoV-2 in a subject, but these tools do not assess whether the antibodies are protective against infection. Furthermore, these tools can suffer from poor accuracy and may detect cross-reactive antibodies to non-relevant related coronaviruses. Accordingly, new and accessible tools for assessing the degree of protective immune response to a selected pathogen are needed.

In a second and related aspect, there is a need in the art for tools that enable a survey of immune elements present in a subject. In each person, the immunological response to antigens such as pathogenic microbes, environmental elements, and allergens results in a unique, individual response. Certain types of immunoglobulins may be produced, such as: secreted IgA antibodies in the respiratory tract; circulating IgG antibodies, which may take some time to appear following infection; rapidly deployed IgM antibodies; and others. Depending on the type of infectious agent, the site of infection, and its pathological processes, certain immune responses will be more effective than others. Accordingly, in various diagnostic and therapeutic contexts, it would be desirable to identify or assess the presence of immune elements that act as biomarkers of infection and immunity. Currently, clinical evaluation of patient immune responses to stimuli such as allergens or microbial pathogens, requires either the direct detection of the pathogen within a biological sample provided by a patient, or indirect detection of an antibody response (i.e. ELISA or allergy skin test). For pathogens that present in low titers, that sequester in tissues that are difficult to sample, that are transmitted through unidentified mechanisms, or that may be the cause of secondary reactions in a patient after pathogen clearance has been achieved, detection of relevant antibody isotypes and subtypes specific to the antigenic stimuli could provide a more reliable and accurate method for diagnostic testing. Additionally, conventional testing methods such as bulk IgG ELISA's do not detect immunoglobulin subtypes (such as IgG3) or glycosylation patterns which are critical to antibody specificity and function.

Tools for the survey of immune elements in a subject have been investigated. For example, as described in PCT International Patent Application Publication Number WO2020072534, entitled High Specificity and Sensitivity Immunosorbent Diagnostic Assays with Simultaneous Resolution of Multiple Antibody Isotypes, a method for surveying immune elements present in a sample is provided. There remains a need in the art for novel extensions and improvements of these immune survey tools, including the need for a full spectrum evaluation of an immune response of interest using a single assay and platform.

These needs and other important contributions to the art are provided in the present disclosure.

### SUMMARY OF THE INVENTION

In a first aspect, the scope of the invention encompasses a functional assay for evaluating a subject's capacity to mount a protective response against a selected pathogen, such as a viral pathogen. By the novel methods of the invention, the presence, and effectiveness, of neutralizing antibodies against a selected pathogen may be determined. By this functional assay, determining individual immunity, monitoring vaccine efficacy, and other benefits are provided.

In another aspect, the scope of the invention encompasses novel immune element survey methods improve current methodologies and extend their application to novel contexts. For example by these methods, the art is provided with tools for surveying the immunoglobulin profile and the presence of other immune system factors directed to selected antigens from pathogens, allergens, and other sources.

In one aspect, the scope of the invention encompasses a method of determining if a subject has efficacious antibodies against a selected pathogen. In various implementations, the invention provides a means of assessing a subject's protective response to infectious agents such as SARS-CoV-2, influenza, and other viral pathogens.

In another aspect, the scope of the invention encompasses a method of surveying, in an individual subject, the immune elements deployed against a selected pathogen, allergen, or other antigen.

In another aspect, the scope of the invention encompasses methods of assessing the presence of reactive immune factors in a sample, by culturing cells comprising pathogens or pathogen-infected cells in the presence of the sample, wherein changes in the behavior of the cultured cells is indicative of reactive immune factors being present in the sample.

In another aspect, the scope of the invention encompasses methods of assessing the presence of reactive immune factors in a sample, by culturing cells comprising pathogens or pathogen-infected cells and immune cells in the presence of the sample, wherein modulation of the interaction between the cultured cells and immune cells is indicative of reactive immune factors being present in the sample.

In another aspect, the scope of the invention encompasses kits, compositions of matter, and articles of manufacture used in carrying out the various methods of the invention disclosed herein.

In another aspect, the scope of the invention encompasses a method of assessing the efficacy of a vaccine or other immunologic-based treatment in the prevention of infection by a selected pathogen.

In another aspect, the scope of the invention encompasses novel methods of assessing immune response to various vaccination formulations.

In another aspect, the scope of the invention encompasses novel methods of assessing potential therapeutic treatments against a selected pathogen.

The various elements of the invention are described in detail next.

### BRIEF DESCRIPTION OF THE DRAWINGS

FIG. 1. FIG. 1 is a diagram summarizing the combined immune functional assay and immune element profiling assay of the invention.

FIGS. 2A and 2B. FIG. 2A depicts an image from an immune functional assay of the invention as described in Example 1. RBD-coated APC beads were treated with sample from a subject prior to their vaccination against SARS-CoV-2. Beads were then incubated with AF488 labeled bulk IgG detection antibodies. Next, the beads were incubated with ACE2-mCherry expressing HEK293 cells. In this rendering, green fluorescent AF488 coated beads (beads having bound IgG) are depicted as black dots. APC beads without bound IgG express red fluorescence intensity and are rendered in this image as white dots. Here, no bound IgG was detected and all beads are white. Bead entry into cells, mediated by RBD-ACE2 interactions, was high, with the majority of beads (113 of 131) being internalized by cells in this image, an INCUCYTE™ live cell imaging system image taken at 1 hour, 30 minutes incubation time.

FIG. 2B. FIG. 2B depicts an image from the immune functional assay of FIG. 2A performed using a sample from the same subject, after SARS-CoV-2 vaccination. This image was obtained at 1 hour 30 minutes incubation time. A substantial portion of the beads have bound IgG (45 black beads vs. 81 white beads). Beads without bound IgG (white) entered the cells with high efficiency (73 of 81 white beads are observed within cells), while only 7 of 45 beads having bound IgG entered cells, demonstrating the presence of bound immune elements from the sample that disrupt RBD-ACE2 interaction.

FIGS. 3A, 3B, 3C, 3D, 3E, and 3F. FIG. 3A-F depict flow cytometry analysis two dimensional plots of bead channel vs. immune element detection agent fluorescence, from the immune profiling assay described in Example 2. Pre-SARS-CoV-2 vaccination and post-vaccination samples from a subject were separately incubated with RBD-coated beads then exposed to a panel of labeled bulk IgG, bulk IgA, and bulk IgM detection antibodies. FIG. 3A: IgG detection agent fluorescence from pre-vaccination sample; FIG. 3B: IgG detection agent fluorescence from post-vaccination sample; FIG. 3C: IgA detection agent fluorescence from pre-vaccination sample; FIG. 3D: IgA detection agent fluorescence from post-vaccination sample; FIG. 3E: IgM detection agent fluorescence from pre-vaccination sample; FIG. 3F: IgM detection agent fluorescence from post-vaccination sample. Comparing pre-vaccination and post-vaccination IgG binding (FIG. 3A and FIG. 3B), a clear increase in RBD-binding IgG is evident post-vaccination.

FIG. 4A, FIG. 4B, FIG. 4C, FIG. 4D, and FIG. 4E. FIG. 4A-4E depict flow cytometry analysis two dimensional plots from FACS analysis of samples from the immune functional assay of Example 3, performed using plasma from a subject previously infected with SARS-CoV-2. FIG. 4A depicts a forward scatter (FSC) vs side scatter plot, distinguishing beads from cells. FIG. 4B depicts bead fluorescence vs. cell fluorescence, wherein internalized beads and free beads are distinguished. Beads were surveyed with a panel of labeled bulk IgG, bulk IgA, and bulk IgM detection antibodies. FIG. 3C: bead fluorescence vs. IgG detection agent fluorescence; FIG. 3D: bead fluorescence vs. IgM detection agent fluorescence; and FIG. 3D: bead fluorescence vs. IgA detection agent fluorescence.

FIGS. 5A, 5B,5C, and 5D. FIG. 5A-D depict partial well images of cultured Borrelia burgdorferi treated with serum, as described in Example 4. Borrelia burgdorferi treated with samples that had not been previously infected: FIG. 5A: 12 hours; FIG. 5C: 3 days, 23 hours. Borrelia burgdorferi treated with samples that had previously been infected with Borrelia burgdorferi: FIG. 5B: 12 hours; FIG. 5D: 3 days, 23 hours.

FIGS. 6A, 6B, 6C, 6D, 6E, 6F, 6G, and 6H. FIG. 6A-H depict partial well images of co-cultured macrophages and Borrelia burgdorferi treated with human serum, as described in Example 5. Macrophages and Borrelia burgdorferi treated with samples from human subject that had not been previously infected are depicted at two time points: FIG. 6A: PHRODO™ pH sensitive dye staining indicative of phagocytosis at 2 hours, 31 minutes incubation time; FIG. 6B: phase contrast image of the field depicted in FIG. 6A; FIG. 6E: pH sensitive dye indicative of phagocytosis at 1 days, 15 hours; and FIG. 6F: phase contrast image of the field depicted in FIG. 6E. Macrophages and Borrelia burgdorferi treated with samples from human subject that had previously been infected with Borrelia burgdorferi are depicted at the same time points: FIG. 6C: pH sensitive dye indicative of phagocytosis at 2 hours, 31 minutes; FIG. 6D: phase contrast image of the field depicted in FIG. 6C; FIG. 6G: pH sensitive dye indicative of phagocytosis at 1 days, 15 hours; FIG. 6H: phase contrast image of the field depicted in FIG. 6G.

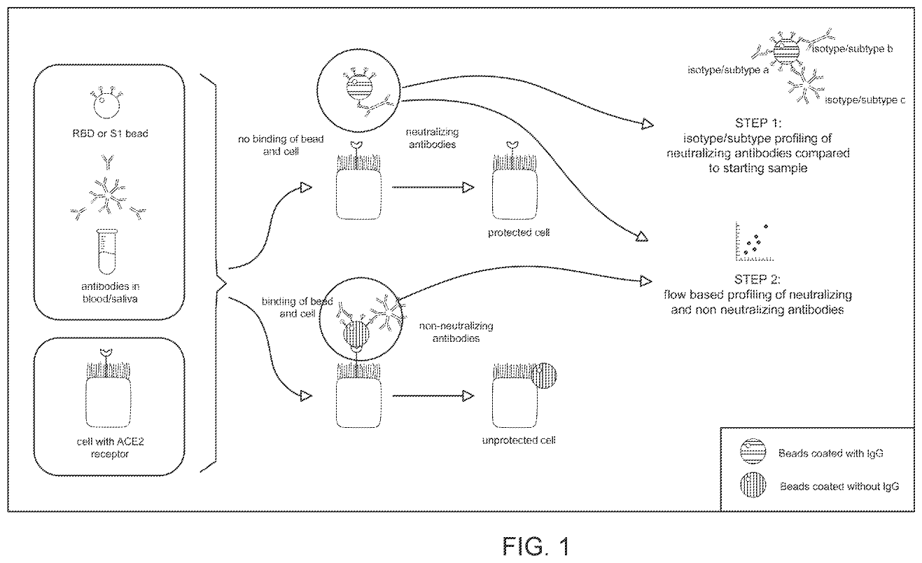

### DETAILED DESCRIPTION OF THE INVENTION

### Part I. Defined Terms

The following terms are referenced and utilized throughout the present disclosure.

As used herein, an “immune element” is any host immune cell, protein, signaling molecule, or other factor that mediates an immune response to an antigen, either specifically or non-specifically. Immune elements will encompass immunoglobulins, i.e. antibodies, including, for example, any of IgM, IgG1, IgG2, IgG2a, IgG2b, IgG3, IgG4, IgA, and IgE immunoglobulins, as well as glycosylation motifs thereof. Immune elements may further encompass cells of the immune system, for example, T-Cells, B-cells, and Natural Killer cells, neutrophils, monocytes, and macrophages. Immune elements may further encompass signaling molecules, for example; cytokines and chemokines that are mediators of immune activity.

“Neutralizing antibodies” means antibodies that inhibit infection by a pathogen. In one embodiment, the neutralizing antibody is an antibody against a viral target. In the case of viral pathogens, the neutralizing antibody may act by binding to target viral antigens, inhibiting the interaction between viral proteins and host receptors, inhibiting viral entry into the cell, and/or inhibiting viral replication within the cell. A subject having neutralizing antibodies against a pathogen may be referred to as having an effective or neutralizing immune response to the pathogen.

“Immune Response Target” (IRT) means a composition of matter which induces, or is capable of inducing, an immune response in a subject. In various implementations, the IRT may be, for example, an antigen, an epitope of an antigen, a pathogen, an allergen, or any other target or putative target of the immune system. In some implementations, the IRT is an antigen comprising a viral protein or receptor-binding fragment thereof, for example, viral attachment proteins that mediate viral interactions with host cells, such as spike proteins and capsid proteins that interact with factors of the host cell plasma membrane.

“Subject” means an individual animal. In a primary implementation, the methods and tools of the invention are directed to human subjects. For example, the subject may be a human subject in need of treatment for a selected condition, or at risk of having the selected condition, the condition being, for example, a pathogenic infection, an allergy, a hypersomnia, an autoimmune condition, or other condition. The subject may be any animal, for example, a human subject, a non-human primate, a mouse, rat, other rodent, dog, cat, cow, pig, horse, or any other animal species. In one embodiment, the subject is a human patient. In one embodiment, the subject is a veterinary subject. In one embodiment, the subject is a test animal.

“Sample” as used herein means any material derived from a subject, for example, as used in the diagnostic methods disclosed herein. Preferred samples are those comprising immune elements, for example, samples comprising antibodies. Samples may comprise, for example, any of blood, serum, saliva, mucus, sputum, urine, sweat, interstitial fluid, spinal fluid, cerebral fluid, tissue exudates, macerated tissue samples, cell culture exudates, etc. Samples may comprise, for example, raw samples, processed samples, self-collected samples, material collected by nasal swab, or a processed sample comprising isolated or concentrated immunoglobulins and/or immune cells. Advantageously, the methods of the invention may be performed with small sample volumes and self-collected samples such as finger prick blood samples, for example, self-collected by a subject and mailed to a clinic for analysis.

An “immune element survey,” as used herein, means an assessment of the presence and/or abundance of two or more selected immune elements in a sample. In a primary embodiment, the immune element survey is achieved by use of an “survey panel” comprising differentially labeled antibodies (or other detection moieties) that bind to specific immune element types, for example, fluorescently labeled antibodies. The survey panel may comprise, for example, antibodies specific to Ig subtypes, Ig isotypes, glycosylation moieties, sialic acid modifications, or other functional modifications.

Antigen Baits.

The methods of the invention are facilitated by the use of what will be referred to herein as “antigen baits.” An antigen bait means a composition of matter comprising one or more IRTs, wherein the IRTs are presented such that they are accessible to immune elements in a sample, particularly antibodies. The antigen bait comprises a platform element and a plurality of IRTs presented thereby. One function of the antigen bait is to provide IRTs to immunoglobulins or other immune elements present in a sample such that the immunoglobulins or other immune elements, if compatible, will bind to or otherwise associate with the IRTs of the antigen bait. In some contexts, such as functional immune assays, the antigen bait serves as a proxy pathogen. In some contexts, the antigen bait provides a vehicle for capturing, separating, and characterizing immunoglobulins or other immune elements that bind to the IRTs thereof.

Immune Response Targets. Each platform element may present numerous copies of IRT moieties, for example, presenting tens, hundreds, thousands, or more IRTs, per bait, e.g., acting as a multivalent IRT entity. In one implementation, only a single type of IRT is presented on each bait platform. In other implementations, two or more types of IRTs may be presented on the same bait. In some implementations, multiple antigens from a selected pathogen are presented by the bait, for example, a spike protein and capsid protein of a selected viral pathogen.

The IRTs may comprise any antigen or other immune response-inducing composition of matter (or putative immune response inducing composition). For example, the IRTs may comprise proteins, peptides, glycoproteins, lipids, glycolipids, polysaccharides, chemical compositions of biotic or abiotic origin, or biologically produced entities (e.g. pollen or spores). In some embodiments, the IRT of the antigen bait is a chemical or other synthetic compound. In one embodiment, the chemical is an adjuvant or adjuvant candidate used in a vaccine formulation. In various embodiments, the chemical may be a chemical composition used, or a candidate for use, in consumer products, for example food additives, packaging materials, plastics, cleaning products, cosmetics, or textiles. In some embodiments, the antigen bait is or is derived from an environmental or synthetic allergen, for example, pollen, spores, dander, dust, peanuts, or lint.

In one embodiment, the antigen bait presents IRTs comprised of multiple materials, for example, a mixture of multiple antigenic materials. Exemplary mixtures include heterogenous microbial mixtures or environmental samples, and materials contaminated with chemical substances.

In a primary embodiment, the IRT of the antigen bait comprise an antigen derived from a pathogen or immunologically reactive epitope thereof. For example, the IRT may comprise a viral protein, such as a viral attachment protein.

In one implementation, the IRT is derived from a coronavirus, such as the SARS-CoV-2 virus, for example, comprising one or more proteins expressed on the surface of the virus, or elements thereof. The IRT may comprise a whole protein or a fragment thereof. In one embodiment, the protein is a coronavirus Spike protein, such as a SARS-CoV-2 Spike protein, or a fragment thereof, for example, a Spike protein 51 subunit, a Spike protein S2 subunit, or a Spike protein receptor binding domain (RBD). In some embodiments, the IRT is a coronavirus (e.g. SARS-CoV-2) N protein, M protein, or fragment thereof.

In one embodiment, the IRT is derived from an Influenza virus, for example, an Influenza A or Influenza B virus. The IRT may comprise one or more influenza virus proteins expressed on the surface of the virus, or elements thereof. The IRT may comprise a whole protein or a fragment thereof. In various embodiments, the protein may be any of: a hemagglutinin (HA) protein; a neuraminidase (NA) protein, an influenza matrix protein 1 (M1), an influenza matrix protein 2 (M2), protein, or an Influenza C hemagglutinin esterase fusion (HEF) protein, or a subsequence or fragment of any of the foregoing. In some embodiments, the selected protein, or subsequence thereof, is selected for maximizing the identification of cross-reactive antibodies. For example, various research groups have demonstrated that the globular head of the HA protein is immunodominant.

In other embodiments, the IRT comprises a protein, or fragment thereof, from a bacteria, yeast, fungus or other microbial species. In various embodiments, the IRT may be derived from Borrelia burgdorferi, for example, flagella and Osp protein thereof, Borrelia miyamotoi, Salmonella spp., Streptococcus spp., E. coli, including enteropathogenic E. coli, enterohaemorrhagic E. coli, enterotoxigenic E. coli, enteroaggregative E. coli, enteroinvasive E. coli, and diffusely adherent E. coli; and other pathogenic or non-pathogenic bacterial species and strains. In some embodiments, the IRT comprises a bacterial flagella protein. Flagella are a class of bacterial structures that are highly conserved amongst species and may on their own elicit an immune response independently of the pathogen they are associated with.

Platform Element. With regards to the platform element, numerous compositions may serve as the platform, including biotic and abiotic entities. In one implementation, the platform comprises a cell. In one implementation, the platform element of the antigen bait comprises a cell, wherein the cell expresses one or more membrane proteins that serve as the IRTs. In one implementation, the platform element of the antigen bait comprises a cell, wherein the cell is functionalized with exogenously supplied IRTs to create the antigen bait. In various embodiments, the cell may comprise a bacterial cell, a yeast cell, an insect cell, a mammalian cell or the cell of any other species. Exemplary mammalian cells include, for example, 293T cells, HeLa cells, CHO cells and other cell lines known in the art. In one embodiment, the cell is a cell of a pathogenic species. In one embodiment, the pathogen is an attenuated pathogen, engineered for reduced virulence or impaired ability to replicate. In one embodiment, the cell is an engineered cell, e.g., engineered to express membrane proteins that serve as IRTs, for example, a non-native protein.

In some embodiments, the platform element of the antigen bait comprises a live cell. In some embodiments, the platform element of the antigen bait comprises a cell that has been infected with live pathogen and then fixed in paraformaldehyde or other fixation agent to eliminate virulence but maintain the structural integrity of proteins. The fixed, infected cell is then used as the antigen bait.

In some implementations, the antigen bait comprises a solid support functionalized with IRTs. The solid support may comprise any configuration, for example, wafers, beads, particles, mesh or porous supporting structures, fibers, tubes, etc. In a primary embodiment, the solid support comprises a bead, for example, a spherical, substantially spherical, or ovoid body. Exemplary bead sizes range from nanoscale to microscale beads. For example, beads having diameters in the range of 1 nm to 50 microns may be used. In various embodiments, the bead size may be about 10 nm, 50 nm, 100 nm, 200 nm, 300 nm, 500 nm, or 1 micron. The composition of the beads may vary. Exemplary materials for the beads include, for example, glass, polymers, ceramics, metal oxides, gold or other metals, and composite compositions, for example, comprising a functional material embedded in a silica or polymer matrix. In one embodiment, the beads are fluorescent beads such as allophycocyanin (APC) beads.

The use of beads or other abiotic solid substrates as antigen baits presents certain advantages with regards to sample collection. It is known in the art that certain sample collection tools contain cytotoxic buffers or preservatives such as sodium azide, excessive salts, and other elements that may make the collected sample harmful to cells, which would impair the ability to utilize cellular antigen baits. Additionally, samples themselves may be toxic, as is the case for undiluted saliva, urine, feces and other sample types. Accordingly, the use of abiotic antigen baits that are not sensitive to such factors is advantageous in that these may be presented to the sample, will collect the relevant immune elements, and then can be washed, for example, washed with buffer multiple times, e.g. 1-5 wash steps, to remove sample or sample collection-associated molecules that might interfere with subsequent cell based functional assays. In this implementation, the assay is not affected by the choice of sample collection techniques and a user may select from any suitable sample collection method that preserves antigen-binding immune elements in the sample.

In another implementation, the solid substrate comprises a magnetic or paramagnetic material, for example, comprising iron oxide or other magnetic materials, to facilitate separations.

In another implementation, the antigen bait of the invention comprises a minimal construct, wherein the platform element is minimized or is absent. In one embodiment, the antigen bait of the invention comprises a bare IRT, or an IRT conjugated to a carrier molecule, fluorescent label, or other functional moiety. In one embodiment, the IRT is a fusion protein comprising an IRT element and other elements such as fluorophores, fluorogenic materials such as horseradish peroxidase or a luciferase element, and/or a peptide affinity tag.

Detection. In a typical implementation, the antigen bait comprises a detectable composition or moiety to facilitate imaging of antigen bait interactions with target moieties, as described below, or to facilitate separation and sorting for immune element surveys, as describe below. In one embodiment, the platform element of the antigen bait is a cell, wherein the cell is engineered to express a label such as a fluorescent protein, for example, GFP, YFP, or RFP. Alternatively, cells may be labeled with an exogenously supplied chemical dye, tag, or fluorophore. In one embodiment, the platform element of the antigen bait comprises a solid substrate wherein the substrate comprises a fluorescent material. For example, the fluorescent bead or other substrate may comprise a fluorescent material such as allophycocyanin (APC). In other implementations, the surface of the bead or other solid support is functionalized with fluorescent moieties.

Viral Antigen Baits and Viral Proxies. In some embodiments, the antigen bait comprises a virus or a viral proxy. In some embodiments, the antigen bait is a virus, comprising a pathogenic strain displaying one or more IRTs of interest wherein the virus has been engineered for reduced virulence such that it is not infectious or disease-causing. In one embodiment, the virus is a pathogenic virus expressing IRTs of interest that has been attenuated by a treatment such as heat, chemical treatment (e.g., formalin or β-propiolactone), high pressure, or other attenuation treatments known in the art.

In some embodiments, the antigen bait is a viral proxy. A viral proxy, as used herein, is a composition and having functional or compositional similarity to selected pathogenic virus, including displaying one or more IRTs of the selected pathogenic virus. The viral proxy may be any of a psuedotyped virus, virus-like particle, or abiotic proxy, as described below.

In one embodiment, the antigen bait is a viral proxy comprising a pseudo-typed virus, also known as a pseudovirus, which is a virus of a minimally virulent or non-virulent type that is genetically engineered to actively display selected IRTs, for example, from a a highly infectious virus strain. When the pseudovirus is exposed to susceptible cells, receptor binding can occur and in some cases, viral entry into the host cell is achieved. As the replication machinery of the infectious virus is not present, propagation of the pathogenic virus of interest is not at risk and therefore pseudoviruses can be utilized in lower biosafety level facilities.

In some embodiments, the antigen bait is a viral proxy comprising a pseudovirus that is derived from the vesicular stomatitis virus (VSV), a negative sense, single-stranded, enveloped RNA virus that belongs to the Rhadoviridae family. VSV is an ideal candidate for manipulation as a pseudovirus because it can be handled in BSL2 and rarely causes disease, for example, as described in Laciotti and Tsai, 2007. To generate a VSV pseudovirus, one or more plasmids containing cloned genes of an IRT protein is used for transfection of eukaryotic cells. Those genes are then transcribed and translated into proteins by the VSV replication machinery and are displayed on the VSV envelope creating a pseudovirus.

In some embodiments, the antigen bait is a viral proxy comprising a VSV pseudovirus expressing and displaying one or more SARS-CoV-2 proteins. Exemplary implementations of SARS-CoV-2 pseudovirus are described in: Li, Q. et al. 2018; Nie, J. et al. 2020, and Case et al., 2020. For example, in one embodiment the antigen bait is engineered such that the Spike protein or RBD thereof of SARS-CoV-2 is expressed on the plasma membrane of the VSV. The VSV is may be further modified to remove the endogenous glycoprotein required for replication.

In some embodiments, the antigen bait is a viral proxy comprising a pseudotyped lentivirus engineered to express one or more IRT proteins. In some implementations, the lentivirus pseudovirus is capable of binding and entry into susceptible cells, followed by membrane fusion and RNA entry to the cell. In one embodiment, the IRT genes are expressed in combination with a minimal set of lentiviral proteins (Tat, Gag-Pol, and Rev) that collectively self-assemble around reporter RNA, which and may encode for a reporter molecule such as luciferase or green fluorescent protein (GFP). In one embodiment, the pseudotyped lentivirus may encode one or more protein antigens of the SARS-CoV-2 virion, for example, S1, S2, RBD, N or M proteins, as described by Crawford et al. in “Protocol and reagents for pseudotyping lentiviral particles with SARS-CoV-2 Spike protein for neutralization assays.”

In some embodiments, the antigen bait is a viral proxy comprising a Virus Like Particle (VLP) which is composed of self-organizing protein structures or subunits of a virus and may be configured to display one or more selected IRTs, while lacking element required for virus propagation. For this reason, VLPs are safely used in low level biocontainment laboratories.

In some embodiments, the antigen bait is a viral proxy comprising an abiotic particle, such as a bead, wherein the particle has been functionalized with one or more IRTs of a selected pathogenic virus.

In the typical implementation, the viral proxy antigen baits will be detectable. In some cases, the antigen bait is a pseudovirus or otherwise infectious particle engineered to express a fluorescent protein, such as GFP, wherein, upon entry to the host cell, the fluorescent protein is transcribed and the “infected” cell becomes detectable and distinguishable from non-infected cells. In some embodiments, the viral proxy is functionalized with detectable moieties, such as fluorophores or fluorescent proteins.

In one implementation the antigen bait is provided in a lyophilized form and may be resuspended by suitable solutions at the time of performing the assay.

Target Constructs.

Another element utilized in the methods of the invention is what will be termed herein as a “target construct.” The target construct is targeted by a compatible antigen bait, by its presentation of a target molecule having affinity for the one or more IRTs of the antigen bait. Conceptually, in a protective response assessment, as described below, the antigen bait is analogous to a pathogen and the target construct acts as the host cell targeted by the pathogen.

The target construct typically will comprise two elements: a platform element, and a plurality of target molecules presented thereby. An illustrative example is a target construct comprising a cell (the platform element in this case) that expresses or presents a viral receptor (the target molecule).

With regards to the target molecule, this element may comprise any composition of matter that has mutual affinity for a selected IRT of the antigen bait. In a primary embodiment, the target molecule is a host cell viral receptor. For example, the receptor may be a target of viral infection that facilitates viral attachment to or viral entry into a host cell, for example, an extracellular glycoprotein or glycolipid, a cell adhesion molecule, or a receptor protein. Exemplary receptors include:
angiotensin converting enzyme 2 (ACE2), a target of coronaviruses such as SARS-CoV-2: the target molecule may comprise ACE2, the extracellular domain thereof, or a fragment thereof that binds to the SARS-CoV-2 spike protein RBD, for example, residues 24-42, 79-83, and 353-357 of the human ACE2 protein;
sialylated glycans or sialic acids (both referred to as “SAs”), a target of many viruses such as influenza viruses, enveloped coronaviruses, nonenveloped reoviruses, and polyomaviruses; for example, the target molecule may comprise a sialylated glycan or sialic acid viral target, such as 5-N-acetyl neuraminic acid (Neu5Ac), α2,3-linked SA, α2,6-linked SA, α2,8-linked SA, a gangliosides, such as a mono-, di-, tri-, or tetra-sialogangliosides, or an asialo, a, b, or c series ganglioside;
a cell adhesion molecule, the target of various viruses; for example, the target molecule may comprise an integrin, such as αvβ3, a cadherin, a selectin, an immunoglobulin superfamily (IgSF) receptor, CD4, junctional adhesion molecule A (JAM-A), or coxsackie adenovirus receptor (CAR);
a phosphatidylserine (PtdSer) receptor, the target of certain enveloped viruses, such as the Zika virus, Marburgvirus, dengue virus, Filovirus, and flavivirus; for example, the receptor may comprise a PtdSer receptor such as a MERTK family receptor tyrosine kinases (TAM), a T-cell immunoglobulin and mucin domain (TIM), TYRO3 receptor tyrosine kinase, or an AXL receptor tyrosine kinase.

In some embodiments, the target molecule comprises a moiety that has binding affinity for the IRT, for example, an antibody or antigen-binding fragment thereof, for example, a fluorescently labeled antibody, an aptamer, or a ligand of the IRT.

The target construct will comprise a platform element that expresses or otherwise presents the one or more selected target molecules (e.g. receptors). In a primary embodiment, the platform element is a cell, for example, a mammalian cell or other cell type engineered to express the one or more selected receptor molecules. Exemplary cells include HEK293 cells, HeLa cells, CHO cells, and others. In some implementations, the cell is a cultured or explanted host cell, e.g. a cell of the type normally infected by a pathogen.

In one implementation, the platform element of the target construct comprises a solid substrate, such as a bead, wherein the solid substrate is functionalized with a plurality of the one or more target molecules.

The selected platform (e.g. cell or bead) will typically comprise a detectable label, for example, a fluorescent protein, which enables the target construct to be imaged. In the case of a cellular platform element, the cell may be engineered to express a fluorescent protein or other detectable species. In the case of a solid support, the substrate may be functionalized with fluorescent proteins or other labels.

In some implementations, the platform element of the target construct is omitted and the target construct comprises only the target molecule, for example, a fluorescently labeled receptor or an antibody against the selected IRT(s) of the antigen bait.

### Part II. Functional Immune Assays

In one aspect, the scope of the invention encompasses methods of assessing a subject's capacity to mount a protective response against a selected pathogen, such as a virus. In these assays, an antigen bait representative of a selected pathogen is used in combination with a complementary target construct. The antigen bait will comprise an IRT such as a viral docking protein or receptor binding domain of the pathogen and the target construct will present a target of the selected IRT(s) of the antigen bait. The antigen bait and target construct will be configured such that they will engage in what will be termed herein as an “interaction,” mediated by the mutual affinity of the antigen bait's IRT and the target construct's target molecule. For example, if the antigen bait is a bead presenting a SARS-CoV-2 spike protein, and the target construct is a cell presenting ACE2, the affinity of the spike protein for ACE2 will induce an interaction between the bait and target construct. An interaction, as used herein means any physical interaction between the two elements, including, for example:
endocytosis of the antigen bait by the receptor construct;
binding of the antigen bait to the target construct, e.g. formation of a strong or stable bond such as a reversible or covalent bond; or
an association between the antigen bait and the target construct, e.g. physical proximity or tropism of the antigen bait to the target construct.

The antigen bait and target construct will be configured such that their interaction is detectable. In a primary embodiment, the antigen bait, the target construct, or both the antigen bait and the target construct are labeled, for example, by different fluorescent moieties, and are configured such that the interaction between the two modulates the detectable signal(s) of the elements in a way that is measurable. For example, as demonstrated herein, an antigen bait comprising a fluorescent protein will produce an altered signal when the bait is translocated into the cell by endocytosis. The detectable modulation of signal may comprise a of reduction in the amplitude of the signal, a change in the peak wavelength of the signal, or generation of a novel signal by the combined fluorescence of the antigen bait and target construct.

The interaction may be monitored by any number of imaging modalities compatible with the selected detection moiety of the antigen bait and/or target construct, for example, fluorescence microscopy in the case of fluorescent moieties.

The general immune function assessment of the invention encompasses the following process: a method of assessing whether a subject has neutralizing antibodies against a selected pathogen, the method comprising the steps of:
obtaining a sample from the subject, wherein the sample comprises antibodies;
contacting antigen baits to the sample, wherein the antigen baits comprises one or more IRTs derived from the selected pathogen and incubating the antigen baits and sample under conditions suitable for antibody binding to the one or more IRTs of the antigen bait;
exposing the antigen bait to a target construct, wherein the target construct comprises a target moiety having affinity for the IRT(s) of the antigen bait; and
measuring the interaction between the antigen baits and the target moieties,
wherein, if the interaction between the antigen baits and the target construct is inhibited by exposure to the sample, the subject is deemed to have neutralizing antibodies against the pathogen.

In a primary embodiment, following incubation of the antigen baits and samples, a labeling step is performed, wherein one or more detection elements for the detection of bound immunoglobulins is presented to the baits. For example, a fluorescently labeled antibody to bulk IgG may be used, as described in the Examples. By this step, it can be assessed whether there are immunoglobulins in the sample that have bound to the antigen baits. The inventors of the present disclosure have determined that in some instances, the baits are not consistently labeled. For example, in assays comprising bead antigen baits, even in the presence of antibodies to the antigen of the bait, not all beads will be labeled.

The foregoing labeling step may be omitted. However, the step of labeling of immunoglobulins bound to the bait prior to performing the functional assay presents various advantages. By identifying those baits with bound immune elements and those without bound immune elements, or having low binding, the baits without bound immunoglobulins can be excluded from the analysis, increasing resolution and signal-to-noise for the assay. In some cases, where none or very few baits are found to have bound immunoglobulins, the user of the assay may decide to skip the subsequent functional assay, saving time and reagents. In some cases, where none or very few baits are found to have bound immunoglobulins, the user may recycle the baits for use with another sample.

Pre-screening baits for bound immune elements may also be used to screen a large number of samples at once to ensure positive samples are present before performing the functional analysis. For example, a plurality of samples can be pooled, exposed to the baits under conditions suitable for binding, and detection of bound immune elements performed. If no baits are found to have bound immunoglobulins, the pool of samples is deemed to contain none or very few positive samples and further functional or immune element survey analysis steps may be skipped. In one embodiment, the sample comprises a pooled sample derived from multiple subjects

In the method of the invention, following incubation with the sample, the antigen baits, optionally labeled for binding immunoglobulins, are presented to the selected target construct and the interaction between baits and target constructs is monitored. Disruption of baseline interaction is indicative of protective antibodies or other immune elements being present in the sample. for example, if the interaction is endocytosis of a bead antigen bait by a cellular target construct, a reduction in such endocytosis is indicative of protective antibodies binding to the baits and disrupting the antigen-target interactions. The premise of this method is that for samples derived from a subject having protective antibodies against the selected IRT(s), such antibodies will bind to the IRT(s) of the antigen bait, and when the antigen bait is subsequently exposed to the target moiety, the prior antibody binding will sterically block IRT access to the target moiety of the target construct, inhibiting the interactions between the antigen bait and the target moiety. An illustrative example is provided by the following exemplary embodiment:
the antigen bait comprises a viral proxy such as a bead, pseudovirus, virus like particle, or other composition which presents an IRT comprising the spike protein of SARS-CoV-2, or RBD thereof;
the antigen bait further comprises a fluorescent moiety;
the target construct comprises a cell expressing ACE2,
wherein, the antigen bait is capable of entering the cell by interactions between the spike protein of the antigen bait and the ACE2 of the cell;
wherein, cell entry by the antigen bait is detectable by modulation of the antigen bait's fluorescent signal;
wherein, if the antigen bait is exposed to a sample comprising protective antibodies against the IRT thereof, such antibodies will bind to the spike protein IRT elements of the antigen bait, such that its ability to dock with ACE2 is impeded, reduced, ablated, or otherwise inhibited.

In one implementation, the assay elements are configured such that the antigen bait is a viral proxy presenting one or more IRTs comprising a viral docking protein, the target construct is a cell presenting a receptor of the viral docking protein, and the interaction between the antigen bait and target construct comprises the antigen bait entering the cell by endocytosis or other translocation mechanism. The scope of the invention encompasses any viral docking protein and viral receptor pair that mediate cell entry. In illustrative embodiments, the assay may be configured as follows:
the antigen bait comprises a viral proxy presenting a coronavirus spike protein, in various embodiments comprising a SARS-CoV-2 spike protein, an S1 or S2 subunit thereof, or a receptor-binding domain thereof; and the target construct comprises a cell expressing ACE2;
the antigen bait comprises a viral proxy presenting an influenza HA protein, NA protein, or a combination of HA and NA proteins; and the target construct comprises a cell expressing a sialic acid composition;
the antigen bait comprises a viral proxy presenting an HIV gp 160 protein; and the target construct comprises a cell expressing CD4,CCR4, and CXCR5;
the antigen bait comprises a viral proxy presenting a herpes simplex virus 1 glycoprotein D; and the target construct comprises a cell expressing herpesvirus entry mediated A (HveA) protein;
the antigen bait comprises a viral proxy presenting a poliovirus capsid shell VP1, VP2, or VP3 protein; and the target construct comprises a cell expressing CD155;
the antigen bait comprises a viral proxy presenting a Rhinovirus capsid shell VP1, VP2, or VP3 protein; and the target construct comprises a cell expressing intracellular adhesion molecule 1 (ICAM1); or
the antigen bait comprises a viral proxy presenting a Zika virus glycoprotein E capsid shell VP1, VP2, or VP3 protein; and the target construct comprises a cell expressing intracellular adhesion molecule 1 (ICAM1);

In this configuration, the interaction comprises endocytosis of the viral proxy into the target construct cell. In one embodiment, modulation of the fluorescence of the viral proxy caused by entering the cell provides a measure of the interaction. In other implementation, such as in the case of viral proxies comprising engineered virus or pseudovirus comprising genes for fluorescent proteins, expression of the fluorescent protein in the target construct cell provides a measure of the interaction.

In alternative implementations, the antigen bait does not enter the target construct, but the proximity of the antigen bait to the target construct modulates the fluorescence of one or both elements, providing a measure of the interaction of the antigen bait and target construct. For example, binding or association of a viral proxy with the membrane of a target cell may modulate fluorescence of the viral proxy, or such interactions may be detected by quantification of paired signals, i.e. wherein the viral proxy and target cell are observed to be in proximity.

In the assays of the invention, the interaction between the antigen bait and target construct in the absence of sample may be measured to establish a baseline interaction value. A reduction in interaction caused by exposure to the sample can then be quantified against this baseline.

In one implementation of the, the sample and antigen baits are first incubated in the absence of target construct, for example for a period of minutes to hours, in order to facilitate the binding of antibodies to the antigen bait, if such antibodies are present. Subsequently, the target constructs are presented to the antigen baits. In an alternative implementation, the antigen baits, sample, and target constructs are incubated together in a single step.

The interaction between the antigen baits and target constructs will occur on time scales of minutes to hours. In one implementation, the interaction is monitored for a period of time ranging from 30 minutes to 72 hours, for example, 1 hour, 1.5 hours, 12 hours, one day, two days or three days. Monitoring of the interaction over time may be facilitated by the use of automated imaging systems for monitoring cultured cells, such as the INCUCYTE™ live cell analysis system (Essen Bioscience). Machine learning software may be employed to recognize and distinguish baits, baits labeled for bound immunoglobulins, cells, baits associated with or internalized by cells, and bare culture well surface, in order to quantify interactions, for example, as described in Example 1. In one implementation, FACS or flow cytometry is utilized, wherein following incubation of antigen baits and target constructs, the reaction is lifted (for example, with the aid of cell dissociation agents such as Versene) and then analyzed by flow cytometry, FACS, or like methodologies, wherein antigen baits, antigen baits having bound immunoglobulins, cells without associated baits, baits associated with or internalized by cells, and other combinations of elements may be identified and quantified, as in Example 2.

The antigen baits will be presented the sample under conditions suitable for antibody binding to the antigen bait, if present, and subsequently, the interaction between the antigen bait and target construct. One of skill in the art may, without undue experimentation, adjust the titers and dilutions of antigen bait, sample, and target construct in order to maximize the detection of the interaction. One advantage of the methods of the invention is that very small sample volumes may be utilized. In some implementations, the dilution of sample for use in the functional assays or immune profiling assays of the invention is a ratio of sample to buffer of at least 1:100, at least 1:200, at least 1:300, at least 1:400, at least 1:500, at least 1:600, at least 1:700, at least 1:800, at least 1:900, or at least 1:1,000. For example, in one implementation the sample comprises a blood sample, or plasma or serum derived from a blood sample, wherein the blood sample has a volume of 1-10 μl. In one embodiment, the sample comprises a blood sample, or plasma or serum derived from a blood sample, wherein the blood sample has a volume of 1-3 μl. In one embodiment, the sample comprises 1-10 μl of plasma or serum. In one embodiment, the sample comprises 1-3 μl of plasma serum. In one embodiment, the sample comprises less than 1 μl of plasma or serum. In one embodiment, the sample comprises less than 20 μl saliva. In one embodiment, the sample comprises less than 10 μl saliva.

A measured attenuation of the interaction between the antigen baits and target construct by exposure to sample, compared to the interaction in the absence of the sample, provides an indicator that protective antibodies are present, capable of disrupting and inhibiting pathogen interactions with host cells. The protective response can be expressed numerically (e.g. 70% protective), or categorically (e.g. subject is unprotected vs. protected) by comparing pools of like subjects that are known to be immune (e.g. vaccinated subjects or subjects having been exposed to an illness prior) against non-immune subjects. Classifier models and other statistical tools known in the art may be applied to the data to determine appropriate thresholds. In some embodiments, a protective response is deemed to occur if the degree of inhibition of the interaction between antigen baits and target constructs in the assay is any of: at least 10%, at least 20%, at least 25%, at least 30%, at least 40%, at least 50%, at least 60%, at least 70%, at least 80%, at least 90% or at least 95% inhibited.

The protective assessment assay of the invention may be combined with other diagnostic measures, such as an immune element survey, as described below.

Applications. By the methods of the invention, tools are provided to the art for individual diagnostic and treatment applications as well as larger scale efforts such as tracking population immunity to a pathogen, development of vaccines and vaccine regimes, and development of treatments.

At the individual level, the protective assessment assay of the invention provides a tool to determine if a subject has sufficient immunity against a selected pathogen. In some implementations, the scope of the invention encompasses a method of preventing an infection in a subject by the following process:
by the protective assessment assay of the invention, determining if the subject has protective antibodies against a selected pathogen; and
if the subject is deemed to be unprotected or insufficiently protected, administering a suitable treatment to the subject to prevent infection by the pathogen.

In some implementations, the treatment comprises administration to the subject of a vaccination against the selected pathogen. In some embodiments, the subject is a subject at risk of contracting an infection by the selected pathogen. In some embodiments, the subject is a subject that has been exposed to the selected pathogen. In some embodiments, the subject has been diagnosed as being infected with the selected pathogen. In some implementations, the subject is vaccinated and the treatment is administration of a booster or supplemental vaccination dosage to augment insufficient immune response against the selected pathogen. In some implementations, the treatment is quarantining of the subject. In some implementations, the treatment is instruction to use face masks or other facial coverings. In some implementations, the treatment is administration of therapeutic agents against the selected pathogen to augment the subject's insufficient immune response against the pathogen.

In one implementation, the pathogen is a coronavirus, such as SARS-CoV-2. In some implementations, the subject is a subject that has previously been infected with the coronavirus. In some implementations, it is unknown if the subject has previously been infected by the coronavirus. In some implementations, the subject has previously been vaccinated against the coronavirus and the efficacy of the vaccination is being assessed, for example, the efficacy of the subject's vaccination against a new strain of the coronavirus. For example, in the case of SARS-CoV-2, vaccine formulations currently in use were developed based on the originally discovered Wuhan strain of the virus. Since that time, new variants of SARS-CoV-2 have evolved, including the alpha, beta, delta, and lambda variants, and it is of importance to determine if vaccine agents based on older strains will be effective against newly evolved strains. In some implementations, upon a finding of insufficient protective response in the subject, the subject is administered any of a vaccination, a booster vaccination, quarantine, and a therapeutic agent such as antiviral agent. Exemplary therapeutic agents against coronavirus (e.g. SARS-CoV-2) include remdesivir, interferon, parasetamol, ritonavir, lopinavir, steroids, such as dexamethasone, and anti-SARS-CoV-2 monoclonal antibodies. The treatment may comprise a prophylactic treatment comprising one of the foregoing agents.

At larger scales, the protective assessment assay of the invention provides a tool for developing vaccine compositions, including testing of antigens, adjuvants, and other formulation variables. By the methods of the invention, a vaccination formulation candidate can be tested for its overall ability to induce a protective response in subjects at the population level The protective assessment assay of the invention also provides an important public health tool for monitoring immunity at the population level, for example, for assessing prior infection status, efficacy of vaccination regimes, herd immunity status, and the ability of previously administered vaccinations to prevent infection against newly evolving strains.

Immune Function Assay Kits. The scope of the invention further encompasses assay kits that can be used to perform the foregoing methods. As used herein, an “assay kit” will refer to an aggregated collection of products that can be used to quantify the protective immune response against a selected pathogen in a subject. In one implementation, the assay kit will comprise antigen baits and target constructs. In one embodiment, the antigen baits comprise a viral proxy. In various embodiments, the viral proxy is any of an engineered virus, a psuedovirus, a virus like particle, or a bead or other solid support functionalized with one or more viral antigens. In one embodiment, the bead comprises a coronavirus spike protein or RBD thereof. In one embodiment, the bead comprises a SARS-CoV-2 spike protein or RBD thereof. In one embodiment, the target construct comprises a cell. In one embodiment, the cell comprises a cell expressing one or more viral receptors. In one embodiment, the viral receptor is a coronavirus receptor. In one embodiment, the receptor is ACE2.

The kits of the invention may further comprise elements for sample acquisition and processing. In one embodiment, the kit comprises a nasal or oral swab. In one embodiment, the kit comprises a vessel for saliva collection. The kits may further comprise elements such as reference standards, washing solutions, buffering solutions, reagents, printed instructions for use, and containers. The assay kits of the invention may comprise assay biochips or microfluidic devices for sample analysis. The assay kits may further encompass software, e.g. non-transitory computer readable storage medium comprising a set of instructions for operating a computer program which aids in carrying out the measurement and analysis of antigen bait-target construct interactions.

### Part III. Immune Element Survey Methods

### Overview

In one aspect, the scope of the invention is directed to methods of performing a survey of immune elements in a sample. Evaluating the immune response in a patient is a component of current diagnostic tools used by clinicians, however, no commercially available platforms or services currently offer a full spectrum immune profile from a single sample and analysis process. Currently, functional read-outs of pathogen-host responses are relegated to direct imaging or traditional clinical culturing methodologies, requiring increased time and cost.

By the immune element survey methods of the invention, the full spectrum diversity of immune elements in a subject may be evaluated, providing important diagnostic information that can guide therapeutic administration to the subject, as well as aiding larger scale efforts to understand and control disease. Provided herein is a method enabling quantification of all immunoglobulins subtypes and isotypes reactive to a selected antigen or pathogen. The method provides a means of determining immunoglobulin subtypes and/or isotypes reactive to the selected antigen or pathogen by use of a single a sample. The method may further be used to identify the glycosylation motifs and other features of the immunoglobulins and other immune elements that are generated in a subject in response to a selected antigen or pathogen.

The scope of the invention encompasses a general immune survey method comprising the steps of
providing one or more antigen baits to a sample, wherein the sample putatively comprises immune elements reactive to the IRT(s) of the antigen bait;
incubating the sample and antigen baits under conditions suitable to enable binding of reactive immune elements, if present, to the IRT(s) of the antigen baits; and
contacting the antigen baits with a survey panel of detection elements, wherein the detection elements are configured to detect specific immune element types; and
by the binding of the detection elements to the antigen baits, assessing the presence and/or abundance of reactive immune elements in the sample.

The general method of the invention may be implemented in numerous contexts to provide useful diagnostic information about the immune system of the subject from which the sample is derived.

Antigen Baits.

The immune survey method of the invention are enabled by the use of antigen baits, as described herein. In some embodiments, the antigen bait comprises a live cell, such as a eukaryotic cell, that has been engineered to express one or more antigens of interest, such as microbial proteins. Exemplary cells include 293T cells, yeast cells, insect cells, and others that are readily amenable to genetic transformation, as described herein. Alternatively, the baits may comprise a solid support, such as a bead, functionalized with one or more IRTs of interest, as described above.

In some embodiments, the antigen bait is configured in a minimal format, for example, comprising a protein conjugated to one or more functional molecules, for example, a fluorescent label and/or an affinity tag moiety, In this implementation, the antigen bait does not comprise a cell or bead platform, but is composed only of the antigen plus functional molecules for imaging and sorting.

The antigen baits will comprise detectable materials or moieties to enable detection, tracking, separation, and quantification. In some embodiments, the antigen bait is a cell genetically modified to express one or more fluorescent proteins, such as GFP, YFP, RFP or others known in the art. In some embodiments, the antigen bait is labeled by use of a fluorescently labeled antibody. Alternatively, the antigen baits, e.g. cells or beads, may be labeled with a chemical dye or tag. In some implementations, the detection.

In one implementation, each antigen bait presents only a single type of antigen. In an alternative implementation, the antigen bait will comprise two or more different antigens, for example, providing a target for immune elements in the sample that recapitulates complex biomolecules comprising multiple epitopes.

Antigen baits comprising different antigens, or different combinations and ratios of antigens may be combined in the survey methods of the invention, providing a multiplexed assay. In this implementation, each type of antigen bait is labeled by a different detectable moiety so that baits bearing different antigens may be isolated and separately analyzed for bound antibodies.

The antigen baits may comprise materials or moieties that facilitate separation. In some embodiments, the antigen bait comprises one member of an affinity tag pair to enable sorting by use of the other complementary, member of the affinity-tag pair. Exemplary affinity tags pairs include: polyhistidine tagging systems, e.g. 6-histidine HisTags and nickel, biotin-avidin tagging systems; strepavidin-biotin tagging systems; SpyCatcher-SpyTag systems, SnoopCatcher-SnoopTag systems, DogTag tagging systems; Isopeptag tagging systems; and SdyTag tagging systems.

In some implementations, the antigen bait comprises an oligonucleotide tag, e.g. an oligonucleotide barcode, as known in the art, which enables detection and quantification by sequencing techniques. For example, in the case of an antigen bait comprising a cell, in addition to genes coding for antigens of interest, the cell may be engineered to express oligonucleotide tags that identify the antigens expressed by the cell. Alternatively, antigen baits may be functionalized with exogenously supplied oligonucleotide tags that are subsequently detectable in sequencing methodologies.

Survey Panel.

After incubation of the antigen baits and sample, the antigen baits are surveyed by use of a survey panel. As used herein, “survey panel” refers to a panel of detection agents, wherein each detection agent is configured to selectively bind to a specific immune element. In a primary embodiment, the detection elements of the survey panel are antibodies or antigen-binding fragments thereof that are selective for a specific type of immune element. The antibodies or other detection elements will be differentially labeled, for example by fluorescent moieties, or with differing epitopes for the binding of labeled secondary antibodies. This enables detection and quantification of the different immune elements bound to the antigen bait, if any. In some implementations, the detection moieties (e.g. antibodies to various Ig subtypes) are labeled with oligonucleotide sequences for subsequent detection and quantification by sequencing methods. In some embodiments, the detection elements comprise labels distinguishable by ICP-MS or like measurement. The survey panel will comprise detection elements for the detection of two or more immune element types. In a primary implementation, the survey panel is configured to detect two or more immunoglobulins, for example, IgM, IgG1, IgG2, IgG2a, IgG2b, IgG3, IgG4, IgA, IgD, IgE, bulk IgG, IgA1, IgA2, bulk IgA immunoglobulins, monomeric, dimeric, trimeric, tetrameric, pentameric forms of the foregoing immunoglobulins or antibody glycosylation motifs and modifications of glycosylation motifs.

In some implementations, the survey panel will comprise elements for the detection of glycosylation motifs present on immunoglobulins. Glycosylation motifs mediate the activity and specificity of antibody binding interactions in an immune response. For example, the Fc domain of an immunoglobulin interacts with receptors on immune cells throughout the body. Glycosylation modifications to the Fc domain can impact the type of immune response that is elicited by the corresponding immune cell. Glycosylation of the Fc region of IgG antibody occurs post-translationally on the N-linked glycan at asparagine 297. There are 36 different glycans and six classes of Fc receptors found in human immunoglobulins which leads to an abundance of small changes that contribute significantly to different functions of the immune system, for example, as described in Jennewein and Alter, 2017 The Immunoregulatory Roles of Antibody Glycosylation, Trends Immunol 38: 358-372. For example, the detection elements may be used to detect immunoglobulin glycosylation motifs indicative of a particular disease or clinical phenotype.

Analysis. After detection elements have been deployed to the antigen baits, they may be detected and quantified by any suitable methodology. For example, if the detection elements are fluorescently labeled (or are fluorescently labeled by contact with labeled secondary antibodies), methods such as flow cytometry, CYTOF or other fluorescent sorting platforms are applied to quantify the detection elements bound to the antigen baits. Exemplary platforms include Miltenyi Biotec MACSQUANT TYTO™ Cell Sorter. In one embodiment the method utilizes flow cytometric analysis of fluorescently labeled detection elements that can be sorted by factors such as size, state (for example, as in alive or dead for antigen baits comprising cells), fluorescent colorimetric label, and mean fluorescent intensity (MFI). This allows evaluation of parameters such as cells that are alive or dead, clustering cells or elements, antibodies can be separated by fluorophore excitation, percentages of cell types can be calculated, and from the MFI antibody isotypes and subtypes can be quantified. In other implementations wherein detection moieties are barcoded with oligonucleotides, sequencing platforms, PCR or other suitable tools are used to detect/quantify immune elements in the sample.

In various embodiments the separate or combined assays may be performed using automated instrument where the sample is loaded into the instrument and the instrument completes all steps associated with sample preparation and analysis including, but not limited to pipetting, centrifugation, aspiration, incubation, automated imaging and flow cytometric analysis.

In some implementations, the presence or absence of a specific immune element is determined. By standard methods known in the art, statistical threshold values for determining “present” and “absent” status may be developed for a particular implementation. Additionally, immune elements can be quantified to measure their abundance in the sample, including relative abundance, i.e. ratios.

The sample may be further analyzed by additional methods, for example, assays to detect or quantify immune cells present in the sample or assays to detect signaling molecules such as cytokines, chemokines, and other immune factors.

Immune Element Survey Kits. In one aspect, the scope of the invention encompasses kits for performing the immune survey methods of the invention. In a primary embodiment, the kit will comprise:
antigen baits, comprising one or more antigens of interest; and
a survey panel of detection agents for the detection and/or quantification of selected immune elements bound to the antigen baits.

The kit may further comprise sample collection and processing elements, microfluidic elements for performing the assay, instructions and/or software for performing the analysis and interpreting the results, and other elements of the assay.

### Applications

The foregoing immune survey method and assay kits may be applied in any number of diagnostic, treatment, and research applications, as follows.

Combined Assay. In a primary embodiment, the immune functional assay of the invention is combined with the immune element survey methods disclosed herein. In this combined assay, the presence of protective antibodies in the sample is assessed by the use of antigen baits as described in Part II of the present disclosure, followed by analysis of the reactive antibodies bound to the antigen baits, by the methods described in this Part III of the present disclosure.

The combined assay provides important information regarding the identity of neutralizing or non-neutralizing antibodies present in the subject. for example, in the case of a detected protective response by the functional assessment of the invention, it would be useful to know what types of immunoglobulins, glycosylation motifs, and other immune elements are implicated in the protective response. Likewise, assessing the identity of non-neutralizing antibodies can guide efforts such as the development and improvement of vaccine compositions and formulations. The combined assay may also be used to establish antibody profiles associated with protective and non-protective responses.

Assessment of Reactive Immune Response. In one aspect, the immune survey methods of the invention may be utilized to assess the ability of a subject to mount a reactive immune response against a selected antigen. In this implementation, a sample containing antibodies is obtained from the subject; the sample is presented to antigen baits comprising one or more antigens of interest; and the antigen baits are presented to an immune element survey comprising detection elements for immune elements to profile the population of antibodies reactive against the antigen, if present.

In one implementation, the profile of the reactive antibodies is utilized to determine if the subject has a protective response against a selected pathogen. For example, the antigen bait may comprise antigens of a selected pathogen, and if antibodies reactive thereto fall within an immune element profile associated with a protective response, the subject is deemed to have antibodies protective against the selected pathogen. In contrast to the immune assessment tools disclosed herein, this method does not directly assess protective functions of antibodies, but does provides a single and facile assay to detect immune elements that are indicative and predictive of a protective response.

In one implementation, the profile of the reactive antibodies is utilized to determine if the subject has an adverse response against a selected antigen. If antibodies reactive thereto fall within an immune element profile associated with an adverse response, for example, an IgE dominated response, the subject is deemed to have an adverse reaction against the selected antigen.

In one implementation, the adverse reaction is allergy. In this implementation, the antigen bait comprises a selected allergen or putative allergen and the survey panel comprises detection elements directed to immune elements indicative of allergy. Exemplary immune elements indicative of allergy include, for example, glycosylation of IgE antibodies.

For example, in the context of peanut allergy, it has previously been shown that subjects with and without peanut allergy have different glycosylation motifs of IgE, for example, as described in Shade et al., 2020. Sialylation of immunoglobulin E is a determinant of allergic pathogenicity, Nature 582: 265-270. In one embodiment, the method of the invention is utilized to identify subjects having peanut allergy, wherein the antigen bait comprises one or more peanut allergens (e.g., any of Ara h 1-13) and the survey panel comprises elements for the detection of IgE sialylation, bisecting GlcNAc, and/or bisecting galactose, wherein detection of increased IgE sialyation is indicative of peanut allergy and detection of bisecting GlcNAc or bisecting galactose is indicative of non-allergy.

Diagnostic Methods. The immune element survey methods of the invention provides a tool that may be used in various diagnostic applications. In a general embodiment, the method of the invention may be used to assess whether a subject has an infection, or previously had an infection, by a selected pathogen. In this implementation, a sample from the subject is presented to antigen baits comprising an antigen of the selected pathogen, wherein detection of immune elements directed against the antigen is indicative of current, or previous, infection. This application is especially advantageous in contexts wherein direct detection of the pathogen is difficult or not possible. For example, pathogens that present at very low numbers, pathogens that have long incubation periods, or pathogens that take up residence in tissues that are difficult to sample may be indirectly detected by the detection of immune elements targeting the pathogen. Likewise, the methods of the invention enable detection of prior infections wherein the causative agent is no longer present, or is no longer present in detectable amounts.

In one embodiment, the immune survey method of the invention is applied to diagnose the occurrence of multiple co-infections. Often, subjects may be afflicted with two or more simultaneous infections, presenting ambiguous or confounding symptoms. For example, a subject may be simultaneously infected by two or more different pathogens, or two or more strains of the same pathogen, for example, two or more strains of Influenza, such as A, B, and C strains. In this implementation, the sample is presented to a suite comprising two or more types of antigen baits, each type presenting antigen(s) of a different pathogen or multiple stains of a pathogen. The detection of reactive antibodies to more than one strain or more than one pathogen is indicative of current or past infection by multiple infectious agents, and suitable treatment addressing each pathogen may be applied.

In one embodiment, the multiplex survey method allows examination of antibody cross-reactivity between pathogens to identify potential exacerbations between concurrent or sequential co-infections. Pathogens could be identified that share antigenic features triggering pathological cross reactivity where the immune response triggered to one pathogen generates increased risk to have a pathological immune response to a second pathogen. For example, pathological antibodies generated against bacterial peptidoglycan or flagella could then trigger a pathological (or allergic) response to subsequent bacterial infections with similar epitopes. Additionally, pathogens infected concurrently could lead to pathological cross-reactivity in the immune response. Examples of pathogens that could infect concurrently include, SARS-COV-2 and Influenza, Hepatitis B virus and Hepatitis D virus, Giardia intestinalis and Helicobacter pylori, or tick-borne co-infections including Anaplasmosis, Babesiosis, Bartonella Henselae, Borrelia Miyamotoi, Bourbon virus, Colorado Tick Fever, Ehrlichiosis, Heartland Virus, Mycoplasma, Powassan Encephalitis, Relapsing Fever, Rocky Mountain Spotted Fever, Southern Tick-Associated Rash Illness (START), Tick Paralysis, and Tularemia.

For example, in one embodiment, glycosylation of subject antibodies against SARS-CoV-2 is utilized to diagnose the severity of SARS-CoV-2 infection. As described in Chakraborty et al., 2021. Proinflammatory IgG Fc structures in patients with severe COVID-19. Nature Immunology 22: 67-73, disease severity in COVID-19 patients correlates with the presence of proinflammatory IgG Fc structures. In this implementation, disease severity in a subject is evaluated by presentation of a sample derived from the subject to an antigen bait comprising SARS-CoV-2 antigen(s), such as spike protein or RBD thereof, and the survey panel comprises one or more detection agents for proinflammatory glycosylation motifs of IgG1, including fucosylation and galactosylation moieties. If these are detected, the subject is deemed to be at increased risk of severe symptoms, and a suitable treatment may be administered.

Diagnosis of Hypersomnias and other Sleep Disorders. Numerous sleep disorders such as hypersomnia (excessive daytime sleepiness) and narcolepsy have, or are suspected to have, an autoimmune component. In one implementation, the immune profiling methods of the invention are utilized to diagnose sleep disorders. In a general implementation, the antigen bait will comprise an antigen associated with, or putatively associated with, a sleep disorder. The antigen bait is exposed to a sample, such as serum or blood, cerebral fluid, or other sample type, wherein the sample is provide from a subject having, or suspected of having a sleep disorder. The bait is and then profiled using an immune survey panel comprising detection elements for two or more types of immunoglobulins, glycosylation motifs thereof, or other immune elements. If binding to the antigen bait by immune elements is detected, the subject is deemed to have the sleep disorder associated with the presented antigen. In one embodiment, a panel of multiple antigen baits is utilized, for example, differentially labelled beads, cells, each type presenting a different antigens, such that antigens associated with two or more sleep disorders are presented to the sample, providing a multiplex diagnostic tool for identifying the subject's disorder. This is potentially useful for differentiating the cause of the subject's condition, as many sleep disorders present with similar symptoms.

In various embodiments, the antigen bait may present any of the following proteins, and extracellular domains thereof or immunologically active fragments thereof: streptolysin O and Tribbles homolog 2 (associated with narcolepsy); IgLON5 (associated with anti-IgLON5 disease); Ma2 (associated with Anti-Ma2 encephalitis); Caspr2 (associated with Morvan's syndrome); LGI1 (associated with insomnia); Yo, Ro, Gad65, Tr, amphiphysin, Hu, CARP, and GluR1 (associated with Paraneoplastic cerebellar degeneration); and NMDA (associated with anti-NMDA receptor encephalitis).

The method may encompass the further step of administering a suitable treatment for the identified sleep disorder. Exemplary treatments for sleep disorders include, for example: recommending and training behavioral measures and sleep habit regimes such as scheduled naps, promoting regular sleep-wake times, and avoiding driving or other risky activities. Administration of immunotherapies such as IV-administered immunoglobulins, plasmapheresis, and corticosteroids; administration of amphetamines or other wakeful agents; administration of Pitolisant or other histamine modulators; administration of selective serotonin reuptake inhibitors that promote noradrenergic and serotoninergic activity; and administration of GABA-B receptor agonists.

Assessing IgE Dominant Responses. In some individuals, exposure to a pathogen or other triggering composition results in the production of IgE antibodies, predominantly over, or to the exclusion of, the production of IgG antibodies. A first issue presented by this response is potential missed diagnosis by current diagnostic methods that measure only IgG. A second issue is that IgE may trigger pathological effects. Mast cells bind IgE, triggering significant local histamine release, which further amplifies IgE-mediated immune responses while suppressing IgG-mediated immune responses. Mast cell degranulation upon antigen binding to the mast cell IgE receptor leads to histamine and other allergic factor release, potentially causing extensive tissue damage, and may play a role in many of the signs and symptoms of disease involving pathological immune responses including arthritis (or joint/muscle pain), malaise, fatigue, gastrointestinal disturbances, and cognitive impairment.

Accordingly, in one embodiment, the scope of the invention encompasses an immune element survey for the detection of IgE-mediated conditions, wherein
a sample is derived from a subject suffering from a condition associated with mast cell or histamine activity, for example, a condition selected from arthritis, joint or muscle pain, malaise, fatigue, gastrointestinal disturbances, and cognitive impairment;
the antigen bait comprises an antigen of a selected pathogen;
the survey panel comprises detection elements suitable for the quantification of IgE and IgG antibodies reactive against the selected antigen; and
wherein, if the abundance of IgE relative to the abundance of IgG antibodies exceeds a selected threshold, overproduction of IgE antibodies in response to the pathogen is implicated in the condition of the subject. For example, if the antigen specific IgE:IgG ratio exceeds a selected threshold, for example, 2:100, the subject is deemed to have an overactive production of IgE.

If overproduction of IgE is implicated in the condition, the method may comprise the further step of administering to the subject a treatment that inhibits IgE production, inhibits mast cell activity, inhibits histamine, inhibits H4R, or attenuates IgE or histamine related effects.

Assessing mucosal immunity. In one embodiment, the invention may be used to evaluate a subject's IgA production, for example, dimerized IgA production, to assess a subject' level of protection against aerosol transmissible viruses such as seasonal influenza. Sterilizing mucosal immunity at the site of infection by IgA more effectively prevents infection and transmission of aerosol transmissible pathogens, compared to systemic or non-sterilizing immunity that reduces the severity of disease without preventing transmission. In one embodiment, the immune survey method of the invention comprises obtaining a sample from a subject, such as a mucosal sample, presenting the sample to an antigen bait comprising an antigen of a selected aerosol transmissible pathogen, and contacting the antigen bait with a survey panel comprising detection elements for IgA, such as dimeric IgA, wherein abundance of IgA, for example, dimeric IgA above a selected threshold is indicative of a protective immune response against the pathogen.

This methodology could be applied in the development and monitoring of therapeutic or vaccine efficacy, comparing subject immune profile in response to the mechanism of drug or vaccine administration such as inhalation, nasal spray, intramuscular injection or subcutaneous injection. In one embodiment, the method comprises a method of treatment, wherein, if a deficiency of IgA, for example, dimeric IgA is detected, the subject is administered a treatment to increase IgA production or is administered IgA antibodies against the pathogen.

Assessing Ig Ratios and Isotype Imbalances. In another embodiment, the immune survey method of the invention may be used to identify deficiencies of an immunoglobulin isotype or subtype. For example, the IgG isotype is made up of 4 subtypes, IgG1, IgG2, IgG3 and IgG4, with IgG1 being the most abundant of the four subtypes. Deficiencies in IgG subtypes could lead to insufficient clearance of a pathogen or pathological immune responses. For example, in one embodiment, the antigen bait comprises an antigen of a selected pathogen and the survey panel comprises detection elements for assessing two or more IgG isotypes, for example for the measurement of IgG1:IgG2 ratio. If a deficiency or imbalance of subtypes is detected, the subject may be treated by the intravenous administration of antibodies against the selected pathogen of the deficient subtype(s). For example, in the case of a low IgG2 abundance compared to IgG1, by the administration of IgG2 antibodies. In various embodiments, the assay may be configured with detection elements sufficient to assess ratios of antigen-specific Ig types, for example any of: IgE to IgG ratio, IgE to IgA ratio, antigen specific IgE to total IgE; subtype imbalances between IgG1, IgG3, IgG2, IgG4; or isotype/subtype imbalances between IgG subtypes, IgE and IgA.

In one embodiment, the immune element survey is utilized to assess the abundance antibody isotypes and subtypes in subjects having SARS-CoV-2 infection. For example, as described in Nielsen, et al., 2020 B cell clonal expansion and convergent antibody responses to SARS-CoV-2. Research Square, PREPRINT https://doi.org/10.21203/rs.3.rs-27220/v1, ratios of IgG2/IgG1 in healthy human controls is approximately equal to 100%, whereas in humans ill with COVID19 IgG2/IgG1 was observed to be approximately equal to 26%, demonstrating that isotype subtype ratios can be may be used to separate disease states and may be able to further separate cases based on asymptomatic positive carriers, mild disease and severe disease. In this implementation, a sample is derived from a subject having SARS-CoV-2 infection or suspected thereof, the antigen bait comprises a SARS-CoV-2 antigen (e.g. spike protein or RBD thereof), and the survey panel comprises detection elements for the measurement of IgG2/IgG1 ratio, wherein a lower ratio is indicative of infection or more severe disease state. The method may further encompass the administration of a preventative or therapeutic treatment for COVID-19 if such IgG2 deficiency is detected.

### Part IV. Cultured Cell Assays for the Detection of Reactive Immune Elements and the Diagnosis of Disease

In one aspect, the scope of the invention encompasses the use of cultured cells, such as pathogen cells or proxies thereof, to evaluate patient samples for immune elements.

It is generally known that cells may respond to external stimuli with changes in their appearance, clustering, size, gene activation and function. For example, bacterial cells can sense the presence of nutrients, interacting biomolecules, bacteria (quorum sensing) and other factors and respond to these factors. The inventors of the present disclosure have discovered that cellular behavior may be modulated by the presence of immune elements, and that striking changes in cellular behavior may occur when cells are exposed to immune elements that interact with the cells. Advantageously, the inventors of the present disclosure have determined that certain changes in cell behavior occur in the presence of immune elements that engage with cells, and these changes can be used to provide an indicator of immune element presence and activity in a sample. This provides the art with a facile and inexpensive tool for evaluating parameters such as: current or prior infection by a pathogen, the presence of an effective immune response against the pathogen, and other diagnostic measures.

In a general implementation, the encompasses a method of detecting the presence of reactive immune elements in a sample, comprising the steps of:
cultured cells, comprising a cellular pathogen or a cell infected by a viral or prion pathogen, are incubated with a sample putatively containing immune elements that will react with one or more antigens of the pathogen;
the behavior of the cultured cells is assessed and compared to the behavior of cells cultured with samples that do not contain reactive immune elements; and
wherein, modulation of cellular behavior by the sample is indicative of the sample containing immune elements that are reactive against one or more antigens of the pathogen.

In one implementation, the cultured cells are cells of a cellular pathogen. Exemplary cellular pathogens include bacterial pathogens, fungal pathogens, and pathogenic yeast. In another embodiment, the cells comprise cells, such as host cells, infected with a viral pathogen, prion, or other infectious agent.

Exemplary cellular pathogens include: Borrelia burgdorferi, Borrelia mayonii, Borrelia myomoti, Borrelia hermsii, and other spirochetes include Treponema pallidum, Rickettsia spp., Erhlichia, Babesia, Bartonella, Listeria spp., other infectious spirochetes, Staphylococccus spp., Pseudomonas spp., Salmonella spp., Streptococcus spp., pathogenic forms of E. Coli, Mycobacterium spp.

In one embodiment, the cultured cells comprise a pathogen proxy. A pathogen proxy, in this context, comprises a cell, for example, a non-pathogenic cell such as HEK293 cell, that is engineered to expresses one or more antigens or IRTs of a selected pathogen, for example, a spike protein, a bacterial coat protein, a bacterial flagella, or other immune response target.

In a primary embodiment, the cultured cells are engineered to express a fluorescent protein, such as GFP, that facilitates monitoring their behavior in culture by fluorescence microscopy. In an alternative implementation, the cells do not express a label and are subsequently contacted with cellular detection agents such as dyes or stains to facilitate imaging. In another implementation, the large size of the cells makes imaging possible without the need for labeling or staining. The cells may comprise further genetic modifications, such as modifications to make the cells less virulent or infectious, selectable markers, auxotrophic biocontainment traits, and others.

Cells are incubated with a sample that putatively contains immune elements reactive to the cells. For example, if the cell is a bacterial cell, it may be exposed to a sample that putatively contains antibodies against antigens presented by the cultured pathogen (or antigens produced or presented by cultured cells infected with a viral pathogens).

One or more behaviors of the cultured cells is then monitored, wherein, a change of behavior that diverges from that of control cultures, i.e. cells of the same type not exposed to reactive immune elements, indicates that reactive immune elements are present in the sample.

Any cellular behavior may be selected for monitoring. Exemplary behaviors include, for example, growth or growth rate, growth patterns, cellular morphology, topography of the cells or cultures, expression or secretion of biomolecules, biofilm formation or other aggregation, movement, phagocytic activity, distribution and density in culture conditions where localized clustering occurs in samples containing antibodies against the pathogen.

Monitoring may be achieved by suitable instruments for detection of the selected behavior(s). For example, fluorescence microscopy may be used to monitor growth patterns of cultures. In a primary embodiment, cultured cells are monitored using a continuous live cell imaging system such as the INCUCYTE™ live cell imaging system. In some embodiments, the behavior is monitored in an automated fashion by pattern recognition software. Cells may be monitored for any period of time sufficient to evince the altered behavior, for example, for a period of days.

If a divergent behavior is detected, compared to the behavior of cells not exposed to samples containing reactive immune elements, the sample is deemed to contain reactive immune elements, for example, antibodies that react to antigens presented by a cultured pathogen. The presence or absence of such reactive immune elements provides an indicator of various parameters, for example: prior exposure to or infection by the pathogen or prior exposure to a conserved pathogen element such as flagella; current and ongoing infection by the pathogen; a protective immune response against the pathogen; immune deficiency in the presence of a pathogen (i.e. in the case of a vaccination, some people to not generate an adequate response and this would be a way to evaluate that); effect of immune suppressive drugs used by a subject that are present in a sample on pathogens; the potential use of pharmaceuticals to aid the immune response of subject prior to administering pharmaceutical.

In one embodiment, the method encompasses the additional step of administering a suitable treatment if the subject is deemed to be suffering from a current infection of a pathogen or the persistent effects of prior exposure to a pathogen. For example, exemplary treatments may include the administration of antibiotics, antivirals, or therapies to address symptoms of infection, the treatment being selected according to the standard of care for infection by the pathogen.

Aggregation Assay. In a primary embodiment, the monitored behavior is aggregation. For example, the inventors of the present disclosure have discovered that cultured spirochetes such as Borrelia burgdorferi will form biofilm-like aggregates when cultured in the presence of samples containing antibodies against Borrelia burgdorferi. In contrast, bacterial cells exposed to samples that do not contain reactive antibodies will grow in a normal and unaggregated state. Aggregation may be assessed by any number of measures, including, for example
formation of cellular networks, i.e. the formation of physical connections between cells;
viscosity increases in the culture, for example, measured by means of a viscometer or assessed visually by perturbing the culture and observing surface tension, resistance to deformation, or cohesive movement;
sedimentation of cultured cells to the bottom of the culture well or vessel; and
any other features of a biofilm or cellular aggregation are observed.

In one implementation, the aggregation assay of the invention is performed in liquid suspension culture or using a cellular pathogen species that is not adherent. Exemplary species that do not normally aggregate or adhere include, for example, Borrelia burgdorferi. The assay may also be used with adherent species, such as pseudomonas spp.

If aggregation is observed, it is indicative that immune elements reactive to the cultured pathogen are present in the sample. Without being bound to any particular theory of operation, the aggregation may occur as a result of direct cross linking of cells by antibodies, or alternatively, the aggregation may result from physiological changes in the biology of the cells induced by contact with reactive immune elements in the sample, including antibodies, T-cells, cytokines and chemokines, etc.

In one embodiment, aggregation is indicative that the subject from which the sample was derived has been previously infected by the pathogen. In another embodiment, aggregation is indicative that the subject from which the sample is derived is currently infected by the pathogen. In one embodiment, the aggregation is indicative that the subject from which the sample is derived has a protective immune response against the pathogen. In one embodiment, the aggregation is indicative that the subject from which the sample is derived has an immune response to a conserved element such as flagella of the pathogen.

In one embodiment, the pathogen is Borrelia burgdorferi, the primary causative pathogen of Lyme's disease. Borrelia burgdorferi infection may be difficult to diagnose, with standard ELISA based diagnostics being unreliable. In this implementation, Borrelia burgdorferi cells are cultured, for example, Borrelia burgdorferi cells expressing a fluorescent protein to facilitate visualization. The cells are exposed to a sample, for example, plasma or saliva, from a subject that currently or previous had symptoms of Lyme's disease. The cultured cells are monitored for a period of time, e.g. 1-3 days, and aggregation is assessed by network formation, increased viscosity of the culture media, and/or sedimentation. If aggregation is observed, the subject is deemed to have antibodies against Borrelia burgdorferi, indicative of prior or ongoing infection. The method may comprise the additional step of administering a Lyme's disease treatment to the subject, for example, administration of amoxicillin, cefuroxime or antihistamines.

Pathogen-Immune Cell Interactions. In one implementation, the behavior that is monitored is the interaction of the cultured pathogen, pathogen proxy, (or cultured cells infected with viral pathogen) with immune cells. Immune cells may interact with cultured pathogens by any number of responses, for example, binding to pathogen cells, secretion of cytokines or other signaling molecules, or direct attack of the pathogen, for example, phagocytosis by macrophages or cell killing by killer T-Cells.

The inventors of the present disclosure have advantageously determined that immune-cell pathogen interactions may be modulated by exposure to samples containing immune elements reactive to the pathogen. Accordingly, in one implementation, the scope of the invention encompasses: a method of determining if immune elements reactive to a selected pathogen are present in a sample, comprising
co-culturing a selected immune cell type with a target cell comprising a cellular pathogen or a cell infected by a viral pathogen;
observing a selected interaction between the immune cells and the target cells; and
wherein, if the interaction is modulated, relative to that in co-cultured immune cells and target cells not exposed to samples comprising reactive immune elements; the sample is deemed to contain reactive immune elements against the pathogen.

The immune cells may be any immune cell, for example, any of macrophages, eosinophils, mast cells, neutrophils, dendritic cells, monocytes, lymphocytes, natural killer cells, innate lymphoid cells, for example, purified cultures of such cells, or mixtures thereof. In a preferred implementation, the cells are engineered to express a fluorescent protein to facilitate visualization.

In one embodiment, the selected interaction is phagocytosis of the pathogen by the immune cells. In one embodiment, the immune cells are macrophages. In one implementation, inhibition of macrophage phagocytosis by the sample is indicative of the sample containing immune elements reactive against the pathogen. Without being bound to any particular theory of operation, the inhibition of phagocytosis may be mediated by the formation cellular aggregates, induced by reactive immune elements in the sample, which creates a protective structure that the macrophage cannot as easily eat due to the size and scale of aggregated cells.

Conveniently, phagocytosis may be readily visualized and quantified using fluorescence microscopy or flow cytometry methods known in the art. For example, in one embodiment, phagocytosis may be monitored by methods known in the art, for example, by the use of pH sensitive dyes such as PHRODO™ pH-sensitive dyes. Due to large size of macrophages, in some embodiments, standard microscopy may be used to observe and quantify phagocytosis, for example, dark field or bright field microscopy, wherein labeling or dyes are not needed.

In one embodiment, the pathogen is Borrelia burgdorferi. In one embodiment, the Borrelia burgdorferi is cultured with macrophages and phagocytosis is assessed. If phagocytosis of the Borrelia burgdorferi is inhibited, for example, reduced in rate, slowed, or delayed, by exposure to the sample, the sample is deemed to contain immune elements reactive to Borrelia burgdorferi, for example, antibodies against this pathogen.

Mast Cells. In one embodiment, the cultured cells comprising a pathogen, pathogen proxy, or cell infected by a pathogen are co-incubated with mast cells. In this implementation, the selected interaction that is monitored is mast cell degranulation. In one embodiment, mast cell degranulation induced or enhanced by exposure to the sample is indicative of reactive immune elements against an antigen of the pathogen being present in the sample. In one embodiment, the foregoing assay provides a screening tool for therapeutic agents, for example, an antihistamine or putative antihistamine can be added to the reaction to determine if it is effective in reducing histamine release by the mast cells.

Neutrophils. In one embodiment, the cultured cells (comprising a pathogen, pathogen proxy, or cell infected by a pathogen) are co-incubated with neutrophils. In one implementation, the selected interaction to be monitored is phagocytosis of the cultured cells by co-cultured neutrophils. In one embodiment, inhibition of phagocytosis is indicative of reactive immune elements against an antigen of the pathogen being present in the sample In one embodiment, the selected interaction to be monitored is netosis, e.g. the disintegration of cellular or granule membranes in the neutrophil. In one embodiment, netosis induced or enhanced by exposure to the sample is indicative of reactive immune elements against an antigen of the pathogen being present in the sample.

### Part V. Examples

### Example 1. Functional Immune Assay of Pre-Vaccination and Post-Vaccination Serum Samples

In an experimental demonstration of the invention, an immune functional assay to assess the presence of neutralizing antibodies was developed. In this implementation, the antigen bait was a viral proxy comprising APC beads of the LEGENDPLEX™ kit (BioLegend Inc.). The beads were coated with the Spike Receptor Binding Domain (RBD) protein of the Wuhan-hu-1 (Gene ID: 43740568) strain of SARS-CoV-2. By the APC fluorescence, beads alone produced a distinct red signal. After incubation with antibody containing sample from a patient secondary antibody against human IgG that is labeled with AF488, a green fluorescent color, binds to patient antibody that has bound to the RBD coating on the bead and produced a distinct red and green signal.

The target construct was HEK293 cells engineered to express human ACE2 and also engineered to co-express red fluorescent protein mCherry.

In the absence of antibody containing sample, beads exposed to the cultured cells entered the cell with high efficiency (˜90%). This interaction was presumed to occur by bead RBD binding to cellular ACE2, resulting in endocytosis of the bead by the cell, recapitulating the viral infection process. This interaction was monitored in real time by fluorescence microscopy using the INCUCYTE™ live cell imaging system (Essen Bio), incubated in multi-well plates under 5% CO₂ at 37° C. The analysis software of the instrument is designed such that the system can be trained for image recognition, and was trained to recognize: 1) cells, 2) beads, 3) the plate in absence of cells or beads, and 4) beads inside cells or bound to cells, according to manufacturer's techniques.

The microscopy system was programmed to take a series of images of each individual well at set time-points e.g., every 10 minutes), for a defined period of time (e.g., 4 hours). Images from this Example were obtained at 1.5 hours. Beads that did not enter the cell produced distinct spots of green fluorescence due to sample antibodies that were bound to the bead. Cells without internalized beads produced distinct red fluorescence. Beads unbound by antibody can be recognized by red fluorescence, size and uniformity of shape. When beads that are unbound by antibody entered the cells, the combined fluorescent signals of the beads alone (red) and cells (red) produced a signal of increased fluorescent intensity that is distinct and recognized by the software analysis system.

A first sample comprising blood serum was analyzed. The sample was collected from an adult female subject in 2018 before the start of the COVID-19 pandemic.

Beads were first incubated with sample, prior to being presented to the target cells. Diluted plasma (1:900) equating to a volume of 0.03 uL was combined with APC-RBD beads (2.3 μl bead suspension) and incubated on a rocker in the dark.

After several washes in a buffer containing Fetal Bovine Serum (FBS), the sample was stained with a secondary antibody in fluorescent AF488 against human IgG, enabling visualization and quantification of beads bound by sample specific IgG antibodies.

After secondary antibody staining, and additional washes to remove unbound secondary antibody, the beads were presented to the ACE2-mCherry cells and incubated in the INCUCYTE™ system. After 1 hour, thirty minutes of incubation, bead status was assessed in the wells. Beads that were bound by sample specific IgG and those that did not were counted. Beads that entered the cell were counted, and beads that did not enter the cell were counted.

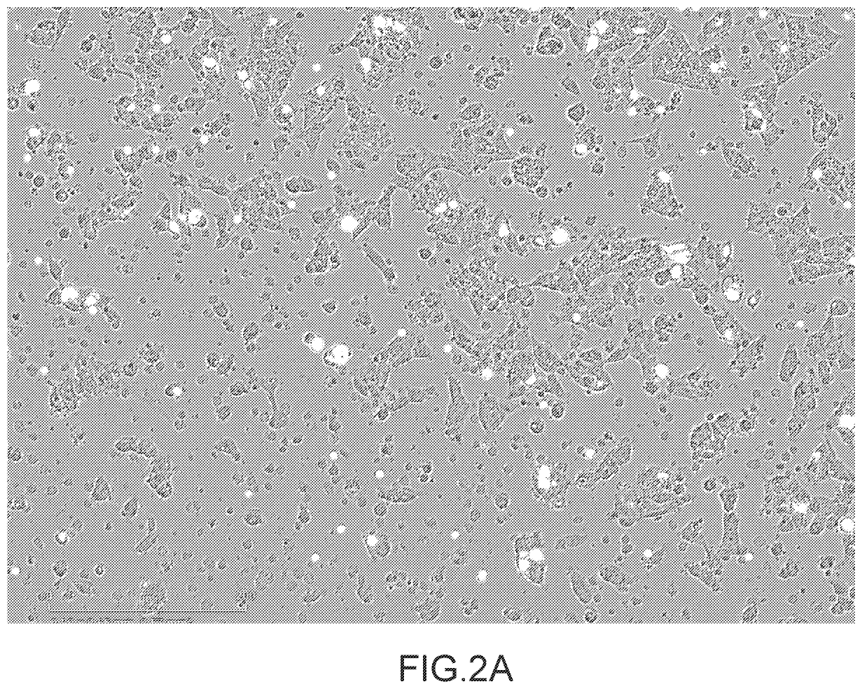

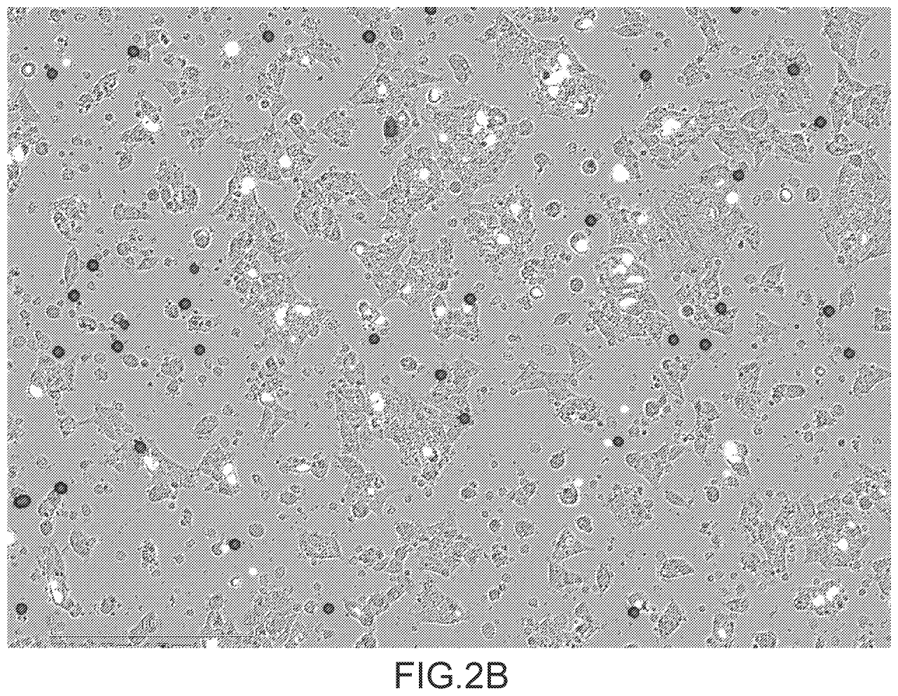

In a representative well, as imaged in FIG. 2A, 131 beads were detected in the image panes selected (4 concatenated images across the center of the well). None of the beads produced an AF488 signal, indicating that no IgG immunoglobulin from the sample was bound to the beads. By detecting the combined fluorescence of the APC bead and mCherry protein of the cell, it was determined that 113 out of 131 (86%) of the beads entered the cells.

A second serum sample from the same donor at a later time point was analyzed. The second sample was collected 35 days following full vaccination against SARS-CoV-2. The subject had received two doses of the MODERNA™ mRNA-1273 vaccine, administered approximately 28 days apart, wherein SARS-CoV-2 spike protein is the antigen element of the vaccine.

The second sample was assayed as above. At 1 hour 30 minutes of incubation, bead fluorescence and location relative to cells was quantified. In a representative well, as depicted in FIG. 2B, 126 beads were detected, 45 of the 126 beads had measurable AF488 fluorescence. Of the beads having detectable bound IgG, only 7 of 45 (15%) entered the cells. Of the beads having no detectable bound IgG, 73 of 81 (90%) entered the cells.

The differing results obtained using the pre- and post-vaccination samples demonstrate the power of the assay to detect neutralizing antibodies specific to SARS-CoV-2 Spike RBD as the beads were blocked from entering the cell by disruption of bead RBD binding to the complementary hACE2 receptor of the cells. In the first sample obtained prior to vaccination, the subject did not produce an immune response specific to RBD of SARS-CoV-2 spike protein, evidenced by the result that no serum antibodies were detected as bound to the RBD-coated beads (0 of 131 showing AF488 fluorescence). Furthermore, after exposure to this sample, the beads entered the cells with high efficiency (86% after 1.5 hours), indicating that exposure to the sample did not inhibit RBD-h ACE2 binding interactions. This is consistent with what would be expected for a subject with no immune response to the RBD peptide.

In contrast, in the analysis of serum obtained following full vaccination against SARS-CoV-2, the subject's sample showed an immune response against SARS-CoV-2 spike protein specific to the RBD peptide. First, 36% of the beads had measurable AF488 fluorescence, indicating that antibodies present in the sample had bound to the RBD-coated beads. Second, this immunoglobulin binding had an inhibitory effect on the bead's ability to enter the cell. Of those beads having measurable IgG binding, only 15% were able to interact with hACE2 and enter the cell, while 90% of the beads (73 of 81 beads) without bound IgG immunoglobulin were internalized. These results indicate that vaccination induced the production of antibodies in the subject that bound SARS-CoV-2 RBD peptide and thereby, preventing RBD-ACE2 binding interactions and reducing cell entry by a factor of 6.

These representative results demonstrate the power of the assay of the invention to measure protective immune responses against a selected antigen.

### Example 2. Immune Element Profiling of Pre-Vaccination and Post-Vaccination Saliva Samples

Saliva samples were collected from the subject of Example 1, by the ORASURE™ collection device (OraSure Technologies). A first saliva sample was collected prior to the subject being vaccinated, and a second saliva sample was collected 35 days following the subject's receipt of a second vaccination dosage, as described in Example 1.

The samples were exposed to an antigen bait comprising BIOLEGEND™ 13×APC beads (BioLegend Inc.) coated with SARS-CoV-2 spike protein RBD peptide, as in Example 1. Sample and beads were incubated on shaker in the dark.

Following incubation, beads were washed in buffer containing FBS and presented to an immune element survey panel comprising three detection elements:
anti-human IgG AF488 antibody for detection of all IgG isotypes bound to the beads, producing a fluorescent emission signal at 488 nm;
anti-human IgA PE-TXRD antibody for detection of all IgA isotypes bound to the beads, producing a fluorescent emission signal at 615 nm;
anti-human IgM PacBlu antibody for detection of IgM bound to the beads, producing a fluorescent emission signal at 455 nm.

Following an incubation period of 20 minutes with secondary antibodies listed above, the beads were washed twice and re-suspended in flow cytometry buffer solution (PBS, 0.5 mM EDTA, 2% FBS). Flow Cytometry analysis was conducted, where the beads are first gated for size on forward scatter (FSC) and side scatter and then subsequently gated by the fluorescence of the bead versus the fluorescence of the antibody (i.e. AF488, PE-TXRD, PacBlu).

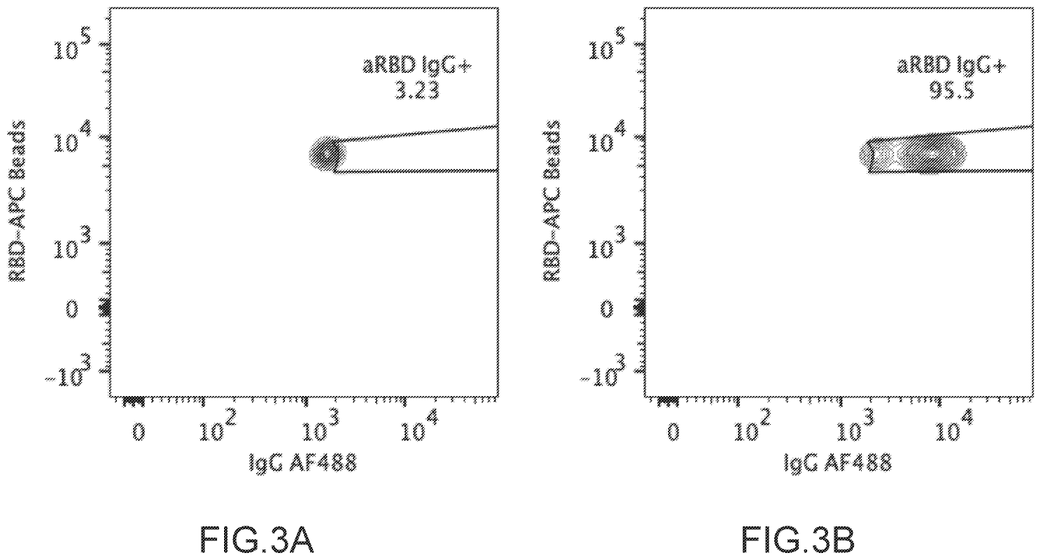

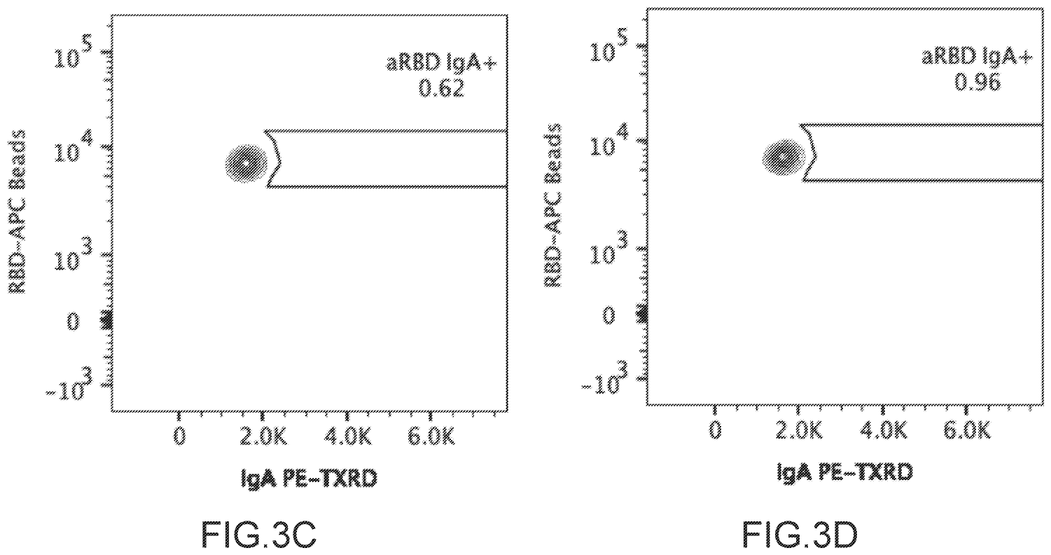

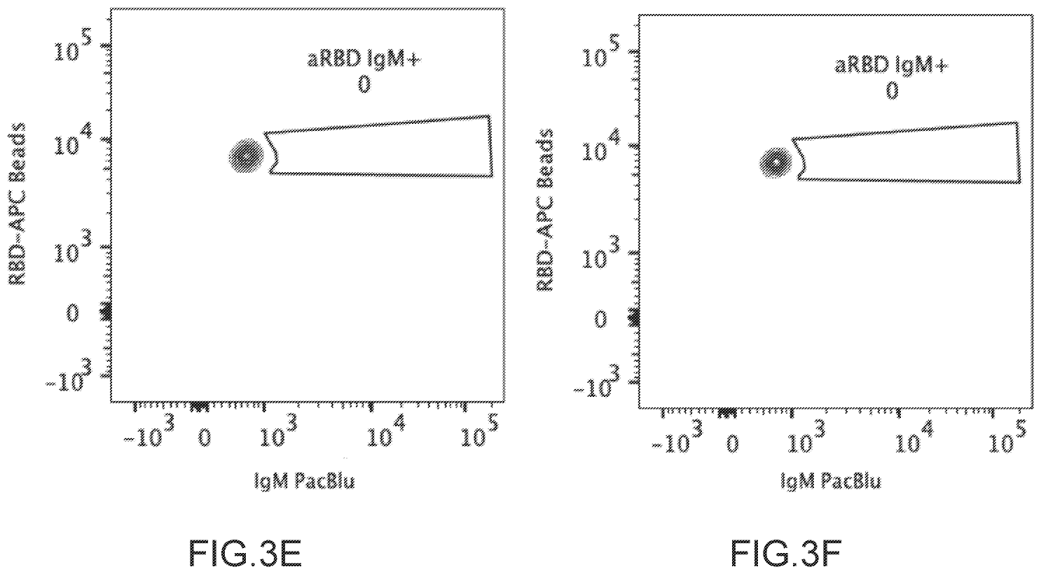

Results are depicted in FIG. 3A-3F as two dimensional plots of the bead channel (Y axis) and antibody channel (X axis). FIG. 3A depicts beads unbound by IgG in the pre-vaccination samples. A single population is evident which sits to the left of the IgG antibody gate (denoted by a solid line drawn in a box shape), demonstrating little to no binding of the IgG detection element to the bead. This indicates that little to no anti-RBD IgG immunoglobulin was in the sample, consistent with the results expected for a subject that was not previously infected by or vaccinated against SARS-CoV-2.

In contrast, as depicted in FIG. 3B, the antigen baits separate into two distinct populations. A first, larger population falls wholly within the IgG antibody gate and a second, smaller population straddles the IgG antibody gate. These results demonstrate that a high abundance of IgG from the sample bound to the RBD-coated beads. These results are consistent with a subject vaccinated against SARS-CoV-2 spike protein and having a robust immune response against the RBD antigen.

Changes in sample IgA binding to the RBD-coated beads pre- and post-vaccination were minimal but measurable, as seen by comparing FIGS. 3C and 3D. No measurable binding of sample IgM to the RBD-coated beads was observed, either before or after vaccination, as seen in FIG. 3D and FIG. 3E which is consistent with the use of a saliva sample.

### Example 3. Functional Assay and Immune Profiling of Convalescent Plasma from COVID-19 Patient

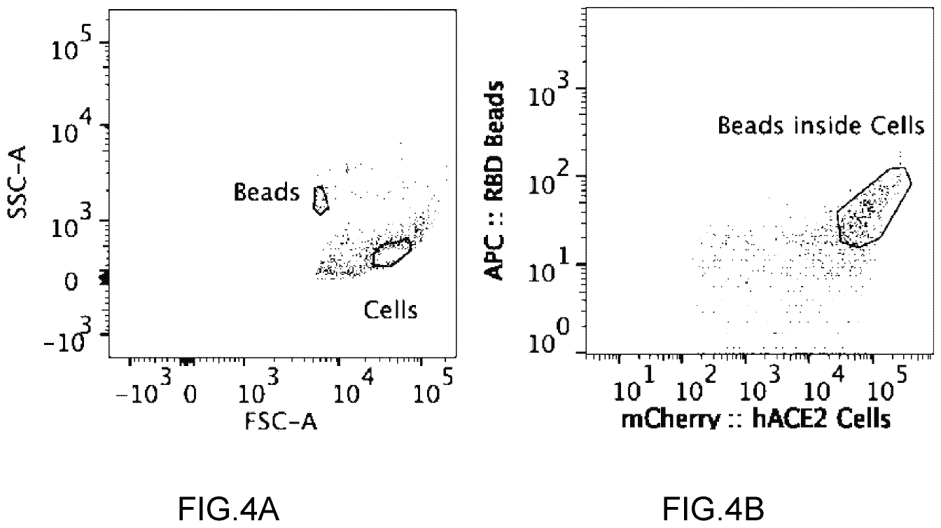

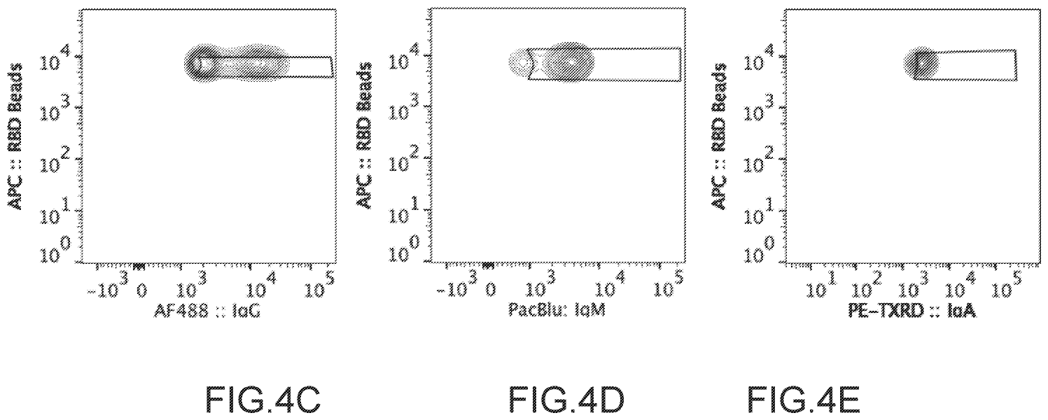

A functional immune assay as configured in Example 1 was performed on a sample comprising plasma from a subject previously infected with SARS-CoV-2, obtained from the Stanford Blood Center (Stanford, CA). After X hours/days of incubation, the reaction contents, including beads and cells of a reaction well were analyzed by flow cytometry. FIG. 4A depicts a forward scatter (FSC) vs side scatter (SSC) plot wherein clear populations of beads and cells can be identified. Samples were subsequently gated by the fluorescence of the bead versus the fluorescence of the mCherry expressing cells, as depicted in FIG. 4B, wherein a clear population of cells containing beads could be distinguished from a substantial population of free beads.

Immunoglobulin binding by all IgG, IgM, and IgA was assessed as in Example 2. APC bead fluorescence vs. detection element fluorescence plots show substantial binding of IgG (FIG. 4C), IgM (FIG. 4D), and IgA (FIG. 4E), as would be expected from a recently infected subject that, post infection, had developed a strong immune response against the spike protein of SARS-CoV-2.

### Example 4. BioFilm Formation in Lyme Subject Serum

In this example, Serum (antibody containing sample from human or animal) from a human subject previously infected with Borrelia burgdorferi (Bb) was provided to a culture of Bb engineered to express GFP.

Cultured Bb was thawed from −80 degrees C. and allowed to expand and propagate for 7 days in borrelia specific media. Cultures were incubated at 37 degrees C. in the dark in sealed culture tubes. The reason for a 7 day preparation of Bb is that the bacterium is in a stressed state initially after thawing where the spirochete is “balled up.” By 24 hours, the spirochetes begin dividing. By day 3 post-thaw, Bb were growing in exponential growth phase. Day 3 post-thaw is the minimum time needed for culture to recover from thaw. By day 7, the culture was in stationary growth phase and ready for use. In the day 7 cultures of Bb, there were approximately 10 million bacteria cells/ml.

In separate experiments, serum from a previously infected human subject was incubated with a 7 day prep of fluorescent Borrelia burgdorferi at a ratio of 1:9 sample:Bb for a final volume of 50 uL in a 96-well flat bottom culture plate. Combined sample and Bb culture was loaded into the INCUCYTE™ live cell imaging system and imaged periodically for several days. In control wells, the Bb culture was combined with serum from human subjects that did not have previous Bb infection.

In the Bb cultures exposed to previously infected animals, the formation of a Biofilm-like matrix occurred rapidly and robustly. Within 24 hours, in the samples from previously infected subjects, a visible network had formed beginning with clumping of spirochetes and developed into an interlinked network having increasing viscosity over time. As Bb are non-adherent, this matrix of cells can be lifted off of the plate using wide-bore pipette tips to assess the viscosity visually. By day 4, Bb culture treated with serum from previously infected subjects formed a thick matrix structure with increasing GFP (green) fluorescence intensity over time, indicating that the Bb were replicating within and remaining in the bacterial matrix.

In contrast, in the Bb cultures exposed to serum from subjects that were not previously infected, the Bb remained in an unaggregated state, dispersed throughout the culture medium and not forming a biofilm-like structure or increasing in viscosity.

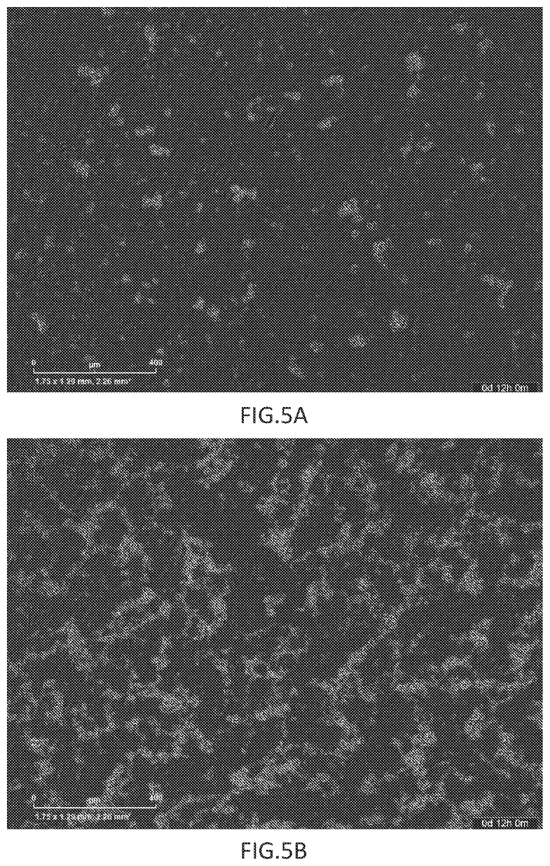

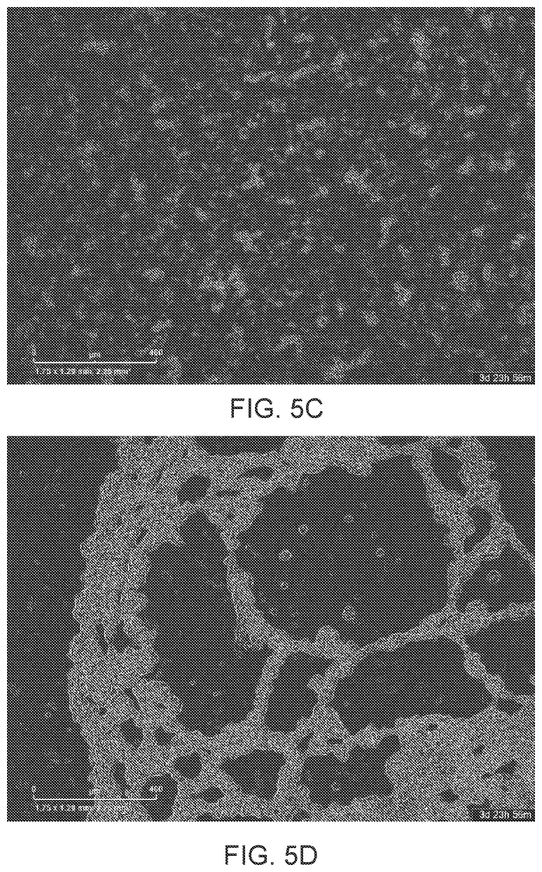

Representative results are depicted in FIGS. 5A-5D, wherein, at each time point, the aggregation of Bb is evident in the cultures exposed to serum from infected subjects (FIG. 5B, 5D), whereas little or no aggregation is observed in the Bb cultures exposed to control serum from uninfected subjects (FIG. 5A, 5C).

### Example 5. Macrophage Response to Serum from Lyme Subjects

Human macrophage cultures were prepared using standard methods. Purified macrophages were seeded at approximately 5000-10,000 cells per well and were incubated with Borrelia burgdorferi from a day 7 prep (previously described). Cultures were subjected to one of two separate samples: A first serum sample from a patient having history of Bb infection. The subject had a self-reported a positive 2 tier test and provided images of bulls-eye rash (erythema migrans). In a control treatment, sample from a human subject not previously infected with Bb was incubated with the macrophage and Bb culture. The cultures were monitored for a period of days, being imaged at regular intervals.

Cultures exposed to the serum from infected and uninfected subjects had strikingly different behavior.

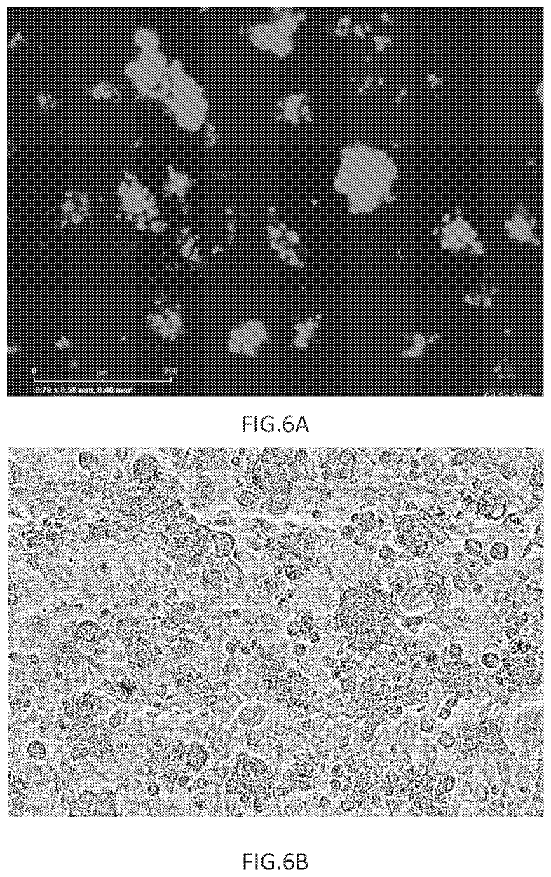

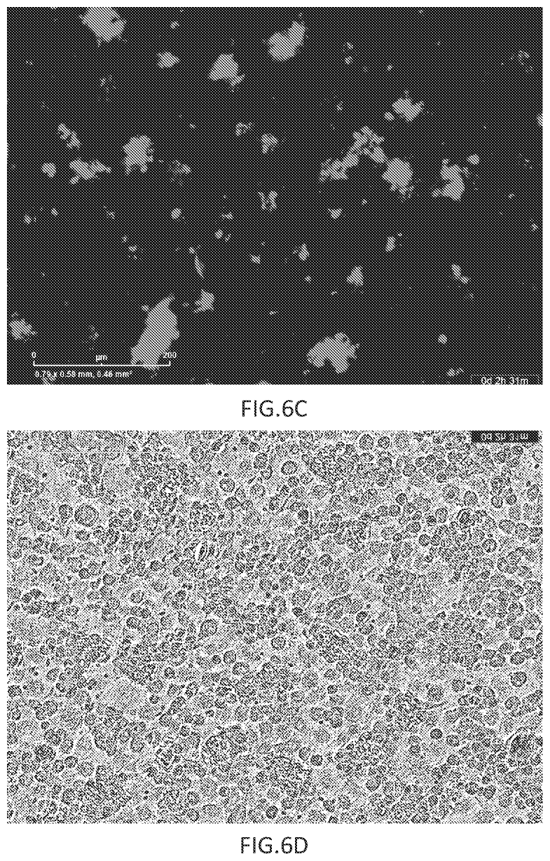

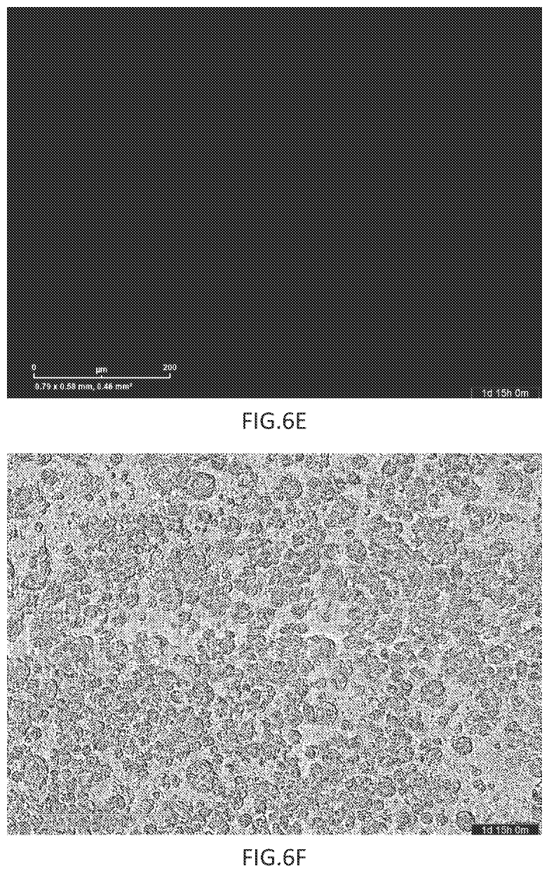

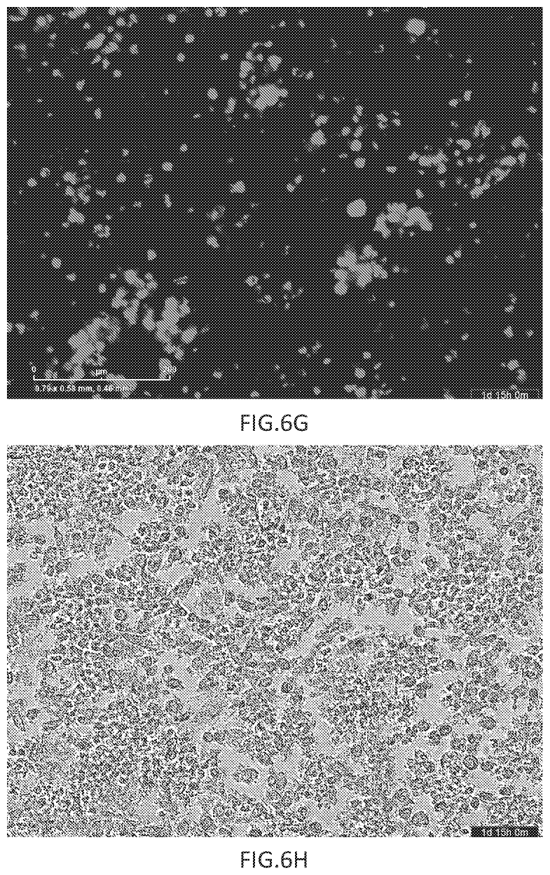

In the presence of a sample from the subject with a history of Lyme disease, the Borrelia in the culture clumped together and phagocytosis activity of the macrophages was delayed or stunted. Meanwhile, in the cultures exposed to serum from a non-infected subject, normal macrophage behavior was observed, with robust phagocytic activity. Representative images are presented in FIG. 6A-6H. Robust phagocytosis was evident in the cells treated with sample from an uninfected subject at the early time point and had largely completed by the later time point (FIG. 6A vs. FIG. 6E). Meanwhile, in cells treated with sample from a previously uninfected subject, phagocytosis was delayed and was still ongoing at a high rate at the later time point (FIG. 6G)

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

1. A method of assessing whether a subject has neutralizing antibodies against a selected pathogen, the method comprising the steps of:
obtaining a sample from the subject, wherein the sample comprises antibodies;
contacting antigen baits to the sample, wherein the antigen baits comprises one or more selected antigens derived from the pathogen;
incubating the antigen baits and sample under conditions suitable for antibody binding to the one or more antigens of the antigen bait;
exposing the antigen bait to a target construct, wherein the target construct
comprises a target moiety having affinity for the one or more antigens of the antigen bait; and
measuring the interaction between the antigen baits and the target moieties,
wherein, if the interaction between the antigen baits and the target construct is inhibited by exposure to the sample, the subject is determined to have neutralizing antibodies against the pathogen.

2. The method of claim 1, wherein
the subject is at risk of contracting the pathogen, has been previously infected with the selected pathogen, or has been vaccinated against the pathogen.

3. The method of claim 1, wherein
the sample comprises blood, plasma, serum, saliva, or nasal swab.

4. (canceled)

5. The method of claim 1, wherein
(a) the antigen bait comprises an abiotic substrate presenting the one or more selected antigens; and
(b) the sample is cytotoxic or comprises cytotoxic elements.

6. The method of claim 1, wherein
(a) the sample comprises 1-3 μl of plasma or serum; and/or
(b) the sample comprises less than 10 μl saliva.

7. (canceled)

8. The method of claim 1, wherein
the sample comprises a pooled sample derived from multiple subjects.

9. The method of claim 1, wherein
the pathogen is a viral pathogen.

10. The method of claim 9, wherein
the pathogen is a coronavirus.

11. (canceled)

12. The method of claim 10, wherein
(a) the antigen bait comprises a coronavirus spike protein, spike protein 51 subunit, spike protein S2 subunit; or spike protein receptor-binding domain; and/or
(b) the antigen bait comprises a virus or viral proxy selected from the group consisting of: an engineered virus; an attenuated virus; a pseudotyped virus; a virus-like particle; or a solid support functionalized with one or more selected viral antigens.

13. (canceled)

14. The method of claim 1, wherein
the antigen bait comprises a solid support functionalized with one or more selected antigens.

15. The method of claim 14, wherein
the solid support comprises a bead.

16.-18. (canceled)

19. The method of claim 1, wherein
the target construct comprises a cell expressing a target moiety having affinity for the one or more antigens of the antigen bait.

20. (canceled)

21. The method of claim 19, wherein
the interaction between the antigen bait and the target construct is endocytosis, phagocytosis, or other internalization of the antigen bait into the cell.

22. The method of claim 1, wherein
if the subject is determined not to have protective antibodies against the selected pathogen, the subject is administered a treatment against the pathogen.

23. The method of claim 22, wherein
the treatment is administration of a vaccine, the administration of a vaccination booster, or a prophylactic treatment.

24. A kit for use in a method of assessing whether a subject has neutralizing antibodies against a selected pathogen, comprising
a plurality of antigen baits, wherein the antigen baits comprise one or more antigens derived from the pathogen; and
a plurality of target constructs, wherein the target constructs comprises target moieties of the one or more antigens.

25.-29. (canceled)

30. A method of identifying a subject's immune elements reactive against a selected antigen, comprising
providing one or more antigen baits to a sample, wherein the antigen bait presents one or more selected antigens and the sample putatively comprises immune elements reactive to the antigen(s) of the antigen bait;
incubating the sample and antigen baits under conditions suitable to enable binding of reactive immune elements, if present, to the antigen(s) of the antigen baits; and
contacting the antigen baits with a survey panel comprising a plurality of detection elements, wherein the detection elements are configured to detect specific immune element types, wherein each detection element type is differentially labeled;
detecting the identity and/or abundance of the detection elements that bind to immune elements bound to the antigen bait; and
wherein, by the detection of the detection elements to the antigen baits, determining the presence and/or abundance of the reactive immune elements in the sample.

31. The method of claim 30, wherein
(a) the survey panel comprises detection elements for the detection of two or more immune elements selected from the group consisting of IgM, IgG1, IgG2, IgG2a, IgG2b, IgG3, IgG4, bulk IgG, IgA1, IgA2, bulk IgA, IgD, and IgE immunoglobulins, monomeric, dimeric, trimeric, tetrameric, pentameric forms immunoglobulins, antibody glycosylation motifs, and modifications of glycosylation motifs;
(b) the survey panel is configured to detect IgE to IgG ratio, IgE to IgA ratio; or the ratio of antigen specific IgE to total IgE levels in the sample; and/or
(c) the survey panel is configured to detect IgG subtype imbalance between IgG1, IgG3, IgG2, IgG4; or isotype/subtype imbalances between IgG subtypes and IgE or IgA.

32. The method of claim 30, wherein
the antigen bait comprises a cell expressing the one or more antigens; or
the antigen bait comprises a solid support presenting the one or more antigens.

33.-34. (canceled)

35. The method of claim 30, wherein
(a) the one or more antigens of the antigen bait comprises a protein of a pathogenic organism; or
(b) the one or more antigens of the antigen bait comprises an allergen or putative allergen.

36.-42. (canceled)

43. A method of determining if immune elements reactive to a selected pathogen are present in a sample, comprising
culturing cells, wherein the cells comprise the pathogen or comprise cells infected by the pathogen;
presenting a sample to the cultured cells and incubating;
monitoring one or more selected behavior of the cultured cells; and
wherein, if the one or more selected behaviors of the cultured cells is modulated, relative to the behavior of like cells not exposed to samples comprising reactive immune elements; the sample is deemed to contain reactive immune elements against the pathogen.

44. The method of claim 43, wherein
the selected behavior is aggregation, wherein aggregation of the cultured cells is indicative of reactive immune elements against the pathogen being present in the sample.

45. The method of claim 44, wherein
aggregation is indicated by formation of cellular networks, increased viscosity of the culture medium, and/or sedimentation.

46. The method of claim 45, wherein
the pathogen comprises Borrelia burgdorferi.

47. The method of claim 43, wherein
the cells are co-cultured with immune cells; and
the one or more selected behaviors comprises an interaction between the cultured cells and the co-cultured immune cells.

48. The method of claim 47, wherein
the co-cultured immune cells comprise any of macrophages, mast cells, neutrophils, eosinophils, dendritic cells, monocytes, lymphocytes, natural killer cells, innate lymphoid cells, for example, purified cultures of such cells, or mixtures of the foregoing.

49. The method of claim 48, wherein
the immune cells are macrophages;
the interaction comprises phagocytosis of the cultured cells by the macrophages or their proximity to each other and distribution within the observed field; and
modulated behavior comprises an inhibition of phagocytosis.

50. The method of claim 49, wherein
the pathogen comprises Borrelia burgdorferi.

51. The method of claim 47, wherein
the immune cells are mast cells; and
the interaction comprises degranulation of the mast cells.

52. The method of claim 47, wherein
the immune cells are neutrophils; and
the interaction comprises netosis or phagocytosis of the cultured cells by the neutrophils.
